(19) United States
(12) Patent Application Publication
(10) Pub. No.: US 2026/0110003 A1
(43) Pub. Date: Apr. 23, 2026

(54) COMPOSITIONS AND METHODS FOR PRODUCTION OF HIGH VALUE CHEMICALS FROM ETHANOL

(71) Applicant: Solugen, Inc., Houston, TX (US)

(72) Inventors: Konrad V. Miller, Sugar Land, TX (US); Toni M. Lee, Missouri City, TX (US); Shuai Qian, Houston, TX (US); Brian Fisher, Houston, TX (US); Philipp Wiemann, Houston, TX (US); Gaurab Chakrabarti, Houston, TX (US); Sean Hunt, Houston, TX (US)

(73) Assignee: Solugen, Inc., Houston, TX (US)

(21) Appl. No.: 18/857,867

(22) PCT Filed: Apr. 27, 2023
(86) PCT No.: PCT/US2023/066326

§ 371 Date: Oct. 18, 2024

Related U.S. Application Data
(60) Provisional application No. 63/335,313, filed on Apr. 27, 2022.
(60) Provisional application No. 63/375,284, filed on Sep. 12, 2022.

(51) Int. Cl. C12P 7/42; C07C 67/31; C12N 9/04; C12N 9/88; C12P 7/18

(52) U.S. Cl. CPC C12P 7/42; C07C 67/31; C12N 9/0006; C12N 9/88; C12P 7/18; C12Y 101/03009; C12Y 101/03013; C12Y 401/01001

(57) ABSTRACT

A chemoenzymatic manufacturing process for the preparation of high value chemicals includes the enzymatic oxidation of ethanol using an oxidizing biocatalyst to form an acetaldehyde intermediate. In addition, the process includes contacting of the acetaldehyde intermediate with a carboligating biocatalyst to form pyruvic acid. Further, the process includes reacting pyruvic acid with ethanol to form ethyl pyruvate. Still further, the process includes contacting the ethyl pyruvate with a metal catalyst and hydrogen to form ethyl lactate. The process also includes hydrolyzing the ethyl lactate to produce lactic acid. Moreover, the process includes contacting at least a portion of the lactic acid with a dehydrating catalyst to form acrylic acid. In addition, the process includes contacting at least a portion of the lactic acid with a metal catalyst and hydrogen to form propylene glycol and n-propanol.

### CROSS-REFERENCE TO RELATED APPLICATIONS

This application is a 35 U.S.C. § 371 national stage application of PCT/US2023/066326 filed Apr. 27, 2023, and entitled, “Compositions and Methods for Production of High Value Chemicals from Ethanol,” which claims priority to U.S. Provisional Application No. 63/335,313 filed Apr. 27, 2022, entitled “Reactor Configuration for the Production of C3 compounds from Ethanol,” and to U.S. Provisional Application No. 63/375,284 filed Sep. 12, 2022, entitled “Compositions and Methods for Production of C3 and C4 compounds from Ethanol,” each of which is hereby incorporated herein by reference in its entirety for all purposes.

### STATEMENT REGARDING FEDERALLY SPONSORED RESEARCH OR DEVELOPMENT

Not applicable.

### REFERENCE TO SEQUENCE LISTING

The instant application contains a Sequence Listing which has been submitted electronically in XML file format and is hereby incorporated by reference in its entirety. Said XML file, created on Nov. 12, 2024, is named “3416-05403_SEQUENCE_LISTING.xml” and is 127 kilobytes in size.

### FIELD

The present disclosure relates generally to the production of higher carbon number compounds from ethanol. More particularly, the present disclosure relates to the chemoenzymatic production of higher value chemicals (HVCs) from ethanol utilizing a novel reactor configuration.

### BACKGROUND

Understanding the conditions in which C—C bonds can be favorably created to make more valuable multicarbon products has been a key commercial goal. Specifically, improved methods for C—C bond formation provide increased opportunities to synthesize useful pharmaceuticals, agrochemicals, and organic materials. Improved methods for the synthesis of C3 compounds such as lactic acid, acrylic acid, n-propanol, and propylene glycol would provide many commercial opportunities.

Lactic acid is an organic acid with the molecular formula CH₃CH(OH)COOH. Lactic acid is the simplest hydroxycarboxylic acid with an asymmetrical carbon atom and has high importance to metabolic pathways. Commercial lactic acid production since 1995 has proceeded through the fermentation approach because it produces a compound with high chiral purity for applications in the food and polymer space. Lactobacillus, Bacillus, and Rhizopus strains are commonly used for producing lactic acid and generate between 85 to 95% yields of the compound from fermentable sugars. Purification challenges for fermentation processes, particularly for products with food applications are considerable. For example, lactic acid is often extracted into an organic phase at low temperatures, then back-extracted into water at high temperature to produce a high-quality product.

Acrylic acid is an organic compound with the formula CH₂═CHCOOH and is the simplest unsaturated carboxylic acid, consisting of a vinyl group connected directly to a carboxylic acid terminus. Acrylic acid is a flammable, volatile, toxic liquid used for polymer production. The compound and its esters readily combine with themselves or other monomers to generate polymers that are used in commercial products. The current method of producing acrylic acid proceeds through direct oxidation of propene via a heterogeneous catalyst. Once product leaves the reactor, it is dissolved in water, extracted with an appropriate solvent, and then distilled to glacial technical grade.

Propanols are three-carbon alcohols that naturally occur in crude fossil oils, fermentation, and decomposition products. 1-Propanol is a primary alcohol with the formula CH₃CH₂CH₂OH (also known as propan-1-ol, 1-propyl alcohol, n-propyl alcohol, and n-propanol). 1-Propanol may also be generated through the hydrogenation of propanal sourced from hydroformylation of ethylene. Production of 2-propanol (also known as isopropyl alcohol, propan-2-ol, 2-propanol, and sec-propyl alcohol) can proceed through the reaction of acetone with hydrogen in the presence of a hydrogenation catalyst.

Propylene glycol has the chemical formula CH₃CH(OH)CH₂OH. The main industrial process for producing propylene glycol is through direct hydrolysis of propylene oxide with water. Propylene oxide and water are combined at a molar ratio of 1:15 at 190° C. and a pressure of about 2 MPa. The reaction generates a 100:10:1 mixture of mono, di, and tripropylene glycol, respectively, which can then be separated by distillation. Applications of this molecule are diverse; propylene glycol is a major component found in unsaturated polyester resins for thermoset composites, food chemistry, food processing equipment, pharmaceuticals, and deicers (i.e., antifreeze). The compound is also found in liquid detergents, plasticizers, paints and coatings, pharmaceuticals, cosmetics, animal feed, tobacco humectants, inks, lacquers, and varnishes.

Acetoin, also known as 3-hydroxybutanone or acetyl methyl carbinol, is a chiral organic compound with the formula CH₃C(O)CH(OH)CH₃. Acetoin is a volatile compound widely employed as flavor and fragrance compound in foods, cigarettes, cosmetics, detergents, chemical synthesis, plant growth promoters and biological pest controls. Acetoin is the main extracellular product of Bacillus subtilis grown on glucose and other fermentable carbon sources and contributes to the aroma and flavor associated with alcoholic beverages. Acetoin is naturally present in foods such as beer, butter and cheese.

2,3-Butanediol, CH₃ (CHOH)₂CH₃, is a butanediol isomer and is considered a significant platform chemical having wide industrial applications. The 2R, 3R stereoisomer is produced by a variety of microorganisms in a process known as butanediol fermentation. 2,3-Butanediol fermentation is the anaerobic fermentation of glucose with 2,3-butanediol as one of the end products. The majority of 2,3-butanediol produced today proceeds from crack gases produced in the refining of fossil fuels. After removal of butadiene, the C4 hydrocarbon fraction, called the C4 raffinate II, is treated with chlorine and sodium hydroxide in water to generate a butene oxide mixture comprised of 55% trans-2,3-butene oxide, 30% cis-2,3-butene oxide, and 15% 1,2-butene oxide. Hydrolysis of this mixture yields butanediols (including 2,3-butanediol) and butene oxides which are separated by vacuum fractionation.

1,3-Butadiene, (CH₂═CH)₂, is a synthetic, colorless gas that is practically insoluble in water and soluble in ethanol, ether, acetone, and benzene. It is used primarily as a monomer to manufacture many different types of polymers and copolymers and as a chemical intermediate in the production of industrial chemicals. The majority of this compound originates from C4 fractions of cracked naphtha and gas oil to ethylene. Butadiene is typically recovered as 2-3.5% of a cracked hydrocarbon feedstock or 25-70% of a typical C4 fraction and must be separated by liquid-liquid extraction or extractive distillation. A minor amount of butadiene is formed from dehydrogenation of n-butane or n-butenes.

2-Butanone, also known as methyl ethyl ketone (MEK) is an aliphatic ketone with the formula CH₃C(O)CH₂CH₃. 2-Butanone is produced in large quantities with nearly half of its industrial use as a component of paints and other coatings because it will quickly evaporate into the air and dissolves many substances. 2-Butanone occurs as a natural product made by some trees and found in some fruits and vegetables in small amounts. Currently, MEK is prepared commercially by the partial oxidation of 2-butanol.

An ongoing need exists for improved methods of producing HVCs such as lactic acid, acrylic acid, propylene glycol, propanol, acetoin, 2,3-butanediol, 1,3-butadiene, and 2-butanone.

### BRIEF SUMMARY OF THE DISCLOSURE

Embodiments disclosed herein include chemoenzymatic manufacturing processes for the preparation of high value chemicals. In an embodiment a chemoenzymatic manufacturing process for the preparation of high value chemicals comprises the enzymatic oxidation of ethanol using an oxidizing biocatalyst to form an acetaldehyde intermediate. In addition, the process comprises contacting of the acetaldehyde intermediate with a carboligating biocatalyst to form pyruvic acid. The process also comprises reacting pyruvic acid with ethanol to form ethyl pyruvate. Further, the process comprises contacting the ethyl pyruvate with a metal catalyst and hydrogen to form ethyl lactate. Still further, the process comprises hydrolyzing the ethyl lactate to produce lactic acid. Moreover, the process comprises contacting at least a portion of the lactic acid with a dehydrating catalyst to form acrylic acid. The process also comprises contacting at least a portion of the lactic acid with a metal catalyst and hydrogen to form propylene glycol and n-propanol.

Embodiments disclosed herein include processes of preparing high value chemicals. In an embodiment, a process of preparing a high value chemical comprises introducing to a plurality of bubble reactors (i) an oxidizing biocatalyst (ii) a carboligating biocatalyst (iii) ethanol, and (iv) carbon dioxide under conditions suitable for the formation of pyruvate.

Embodiments disclosed herein include chemoenzymatic manufacturing processes for the preparation of higher value chemicals from ethanol. In an embodiment, a chemoenzymatic manufacturing process for the preparation of higher value chemicals from ethanol comprises the enzyme catalyzed oxidation and decarboxylation of ethanol to produce pyruvate. In addition, the process comprises subjecting the pyruvate to one or more processes selected from the group consisting of hydrolysis, dehydrogenation, oxidation, distillation, filtration and combinations thereof and recovering one or more products selected from the group consisting of lactic acid, acrylic acid, n-propanol, propanol, propylene glycol or combinations thereof.

Embodiments chemoenzymatic manufacturing processes for the preparation of C4 compounds. In an embodiment, the process comprises the enzymatic oxidation of ethanol using a oxidizing biocatalyst to form an acetaldehyde intermediate. In addition, the process comprises contacting of the acetaldehyde intermediate with a carboligating biocatalyst to form acetoin. Further, the process comprises contacting acetoin with a metal catalyst and hydrogen to form 2,3-butanediol; contacting 2,3-butanediol with a metal catalyst to form 1,3-butadiene; and contacting 1,3-butadiene with a metal catalyst to form 2-butanone.

### BRIEF DESCRIPTION OF DRAWINGS

For a more complete understanding of this disclosure, reference is now made to the following brief description, taken in connection with the accompanying drawings and detailed description, wherein like reference numerals represent like parts.

FIGS. 1-3 are depictions of a plurality of aspects of reaction schemes for the production of 1-propanol, 2-propanol and propylene glycol.

FIG. 4 is a depiction of a reaction scheme for the production of butanone. 1,3-butadiene and 2,3-butanediol.

FIGS. 5-10 depict exemplary reactor configurations for conducting processes of the present disclosure.

FIG. 11 depicts a graph of the enzymatic activities for the indicated galactose oxidase mutants.

### DETAILED DESCRIPTION

Disclosed herein are chemoenzymatic methods for the production of lactic acid, acrylic acid, propylene glycol, and propanol. In an aspect, the methods disclosed herein involve the chemoenzymatic conversion of ethanol to lactic acid, acrylic acid, propylene glycol, or propanol. Hereinafter, lactic acid, acrylic acid, propylene glycol and propanol may be referred to individually as a “C3 product,” or collectively as “C3 products.” Also disclosed herein are chemoenzymatic methods for the production of acetoin, 2,3-butanediol, 1,3-butadiene, and 2-butanone. In an aspect, the methods disclosed herein involve the chemoenzymatic conversion of ethanol to acetaldehyde, which is subsequently converted to acetoin, 2,3-butanediol, 1,3-butadiene, and 2-butanone. Hereinafter, acetoin, 2,3-butanediol, 1,3-butadiene, and 2-butanone may be referred to individually as a “C4 product,” or collectively as “C4 products.”

In an aspect, methods for the production of a C3 product comprise contacting of ethanol with one or more enzymes under conditions suitable to produce an intermediate (e.g., acetaldehyde). Although ethanol is depicted and discussed herein as the substrate, it is contemplated other substrates may be employed. Hereinafter, such contacting of ethanol with one or more enzymes may be referred to as “Stage I.” The intermediate may be further contacted with one or more enzymes and/or one or more chemical catalysts under conditions suitable to produce a C3 compound. Hereinafter, such contacting of the intermediate with one or more enzymes and/or one or more chemical catalysts may be referred to as “Stage 2.”

It is to be understood that while the methods described may be numerically ordered in stages (e.g., Stage 1, Stage 2) for ease of reference, it is not intended to limit the performance of the activities in each stage to a particular order. For example, one or more activities described for a particular stage may be carried out concurrently with one or more activities of another stage whether that “another stage” is designated numerically as being subsequent to or prior to the “particular stage.” Such modifications in terms of the timing of the activities performed in any particular stage may be made by one of ordinary skill in the art with the benefits of the present disclosure.

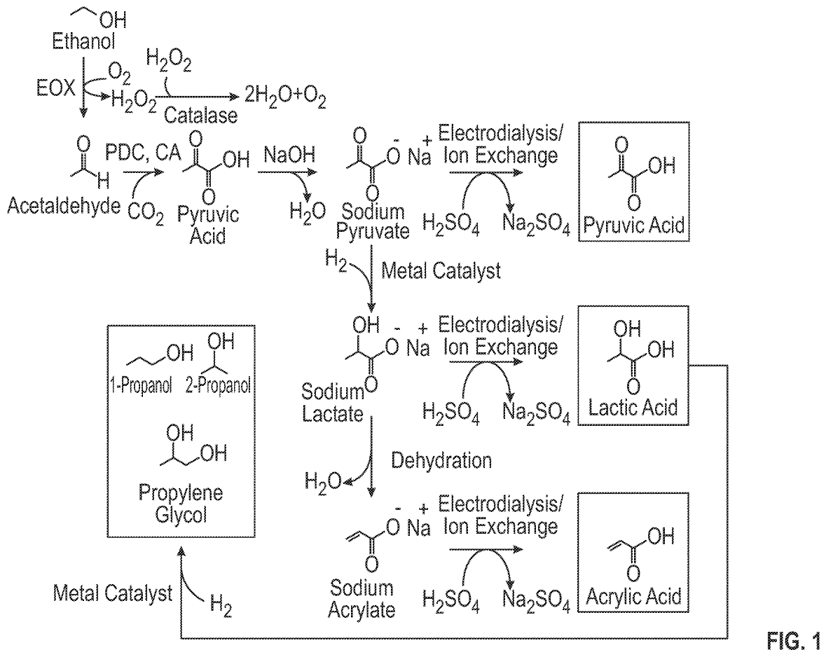

In an aspect, a method for the conversion of ethanol to a C3 compound is generally depicted in FIG. 1. Referring to FIG. 1, a method of the present disclosure may comprise a Stage I where ethanol is contacted with an enzyme capable of catalyzing the oxidation of ethanol to acetaldehyde, alternatively the selective aerobic oxidation of ethanol to acetaldehyde. In one or more aspects, an enzyme for the selective aerobic oxidation of ethanol to acetaldehyde is an ethanol oxidase (EOX). EOX belongs to the family of alcohol oxidases (EC 1.1.3.13) which are enzymes that catalyze the oxidation of primary alcohols to the corresponding aldehydes.

Stage 1 may further comprise contacting of the aldehyde with a pyruvate decarboxylase (PDC) in the presence of carbon dioxide (CO₂) or a source of CO₂ to generate pyruvic acid. Pyruvate decarboxylase is an enzyme (EC 4.1.1.1) that catalyzes the decarboxylation of pyruvic acid to acetaldehyde and is also called 2-oxo-acid carboxylase, alpha-ketoacid carboxylase, and pyruvic decarboxylase.

An equilibrium between pyruvic acid and pyruvate may be established using any suitable methodology such as pH adjustment, electrolysis or ion exchange, as depicted in FIG. 1. In an aspect, the pH adjustment is through addition of any suitable base comprising a monovalent cation. Nonlimiting exemplary suitable bases include sodium hydroxide, which forms a cation pair comprising a single pyruvate anion and a sodium cation.

In one or more aspects, as depicted in FIG. 1, the method further comprises a Stage 2 where pyruvate generated from the reaction of ethanol and EOX is subsequently converted to lactate by partial hydrogenation followed by dehydration to acrylic acid. Alternatively, pyruvate or lactate may be partially hydrogenated to 1,2-propanediol (propylene glycol) or n-propanol.

The Stage 2 portion of methods disclosed herein can proceed via two routes; both catalyzed by a metal catalyst. The first route uses a caustic hydroxide coupled with electrodialysis and ion exchange to co-produce sodium sulfate or gypsum. The second route uses ammonia and esterification to proceed via ethyl ester intermediates (e.g. ethyl pyruvate, ethyl lactate, and ethyl acrylate) without the co-production of salts.

In an aspect, the hydrogenation catalyst comprises a metal catalyst, alternatively a supported metal catalyst. Equilibrium between lactate and lactic acid may be established using any suitable methodology such as pH adjustment, electrolysis or ion exchange, as depicted in FIG. 1. In some aspects, the metal catalyst is chiral and catalyzes the production of predominately or exclusively R-lactic acid; alternatively, predominately or exclusively S-lactic acid.

In an alternative aspect, Stage 2 further comprises dehydration of lactate to form acrylate. Equilibrium between acrylate and acrylic acid may be established using any suitable methodology such as pH adjustment, electrolysis or ion exchange, as depicted in FIG. 1. In some aspects, dehydration of lactate is catalyzed by a metal catalyst, alternatively a supported metal catalyst. The metal catalyst or supported metal catalyst may be the same as or different from the catalyst utilized to facilitate the reduction of pyruvate. In another alternative aspect, Stage 2 comprises hydrogenation of lactic acid in the presence of a metal catalyst or supported metal catalyst and hydrogen to form a compound containing a propyl group. In an aspect, the compound containing a propyl group comprises 1-propanol, 2-propanol, propylene glycol, or combinations thereof. The metal catalyst or supported metal catalyst may be the same as or different from the catalyst utilized to facilitate the reduction of pyruvate via hydrogenation. Any suitable hydrogenation catalyst or dehydration catalyst may be used in Stage 2 of the present disclosure.

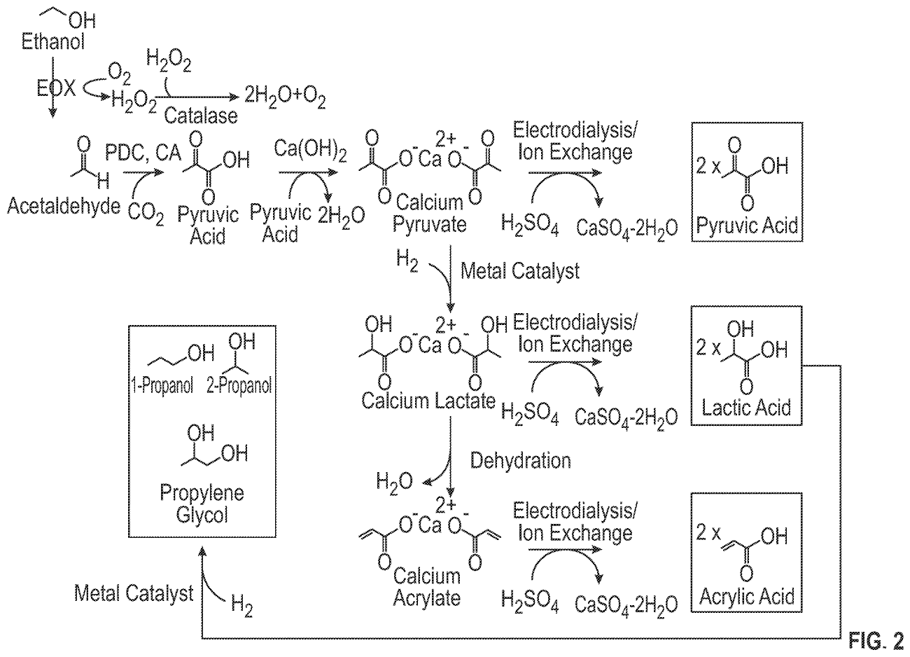

In an aspect, a method for the conversion of ethanol to a C3 compound is generally depicted in FIG. 2. Referring to FIG. 2, a method of the present disclosure comprises a Stage I where ethanol is contacted with an enzyme capable of catalyzing the selective aerobic oxidation of ethanol to acetaldehyde. In an aspect, this enzyme is an EOX of the type disclosed previously herein. Stage 1 may further comprise contacting of the acetaldehyde with a PDC in the presence of carbon dioxide (CO₂) or a source of CO₂ to generate pyruvic acid. Equilibrium between pyruvic acid and pyruvate may be established using any suitable methodology such as pH adjustment, electrolysis or ion exchange, as depicted in FIG. 2. In an aspect, pH adjustment occurs by addition of a base comprising a divalent cation. For example, the base may be calcium hydroxide, which is able to provide two hydroxide ions per calcium atom. The addition of calcium hydroxide to the pyruvic acid results in a cation pair comprising two pyruvate anions and a calcium cation.

The method may further comprise a Stage 2 that includes the reduction of pyruvate catalyzed by any suitable hydrogenation catalyst to form lactate. In an aspect, the hydrogenation catalyst comprises a metal catalyst, alternatively a supported metal catalyst. For example, the hydrogenation catalyst may comprise a supported platinum, ruthenium or copper compound. Equilibrium between calcium lactate (having 2 lactate moieties) and lactic acid may be established using any suitable methodology such as pH adjustment, electrolysis or ion exchange, as depicted in Scheme 2. In some aspects, the catalyst is chiral and catalyzes the production of predominately or exclusively R-lactic acid; alternatively, predominately or exclusively S-lactic acid.

In an alternative aspect, Stage 2 further comprises dehydration of lactate to form acrylate. Equilibrium between calcium acrylate (having 2 acrylate moieties) and acrylic acid may be established using any suitable methodology such as pH adjustment, electrolysis or ion exchange, as depicted in Scheme 2. In some aspects, dehydration of lactate is catalyzed by a metal catalyst, alternatively a supported metal catalyst. The metal catalyst or supported metal catalyst may be the same as or different from the catalyst utilized to facilitate the reduction of pyruvate. In some aspects, the catalyst comprises a zeolite.

In another alternative aspect, Stage 2 comprises hydrogenation of lactic acid in the presence of a metal catalyst or supported metal catalyst and hydrogen to form a compound containing a propyl group. In an aspect, the compound containing a propyl group comprises 1-propanol, 2-propanol, propylene glycol, or combinations thereof. The metal catalyst or supported metal catalyst may be the same as or different from the catalyst utilized to facilitate the reduction of pyruvate.

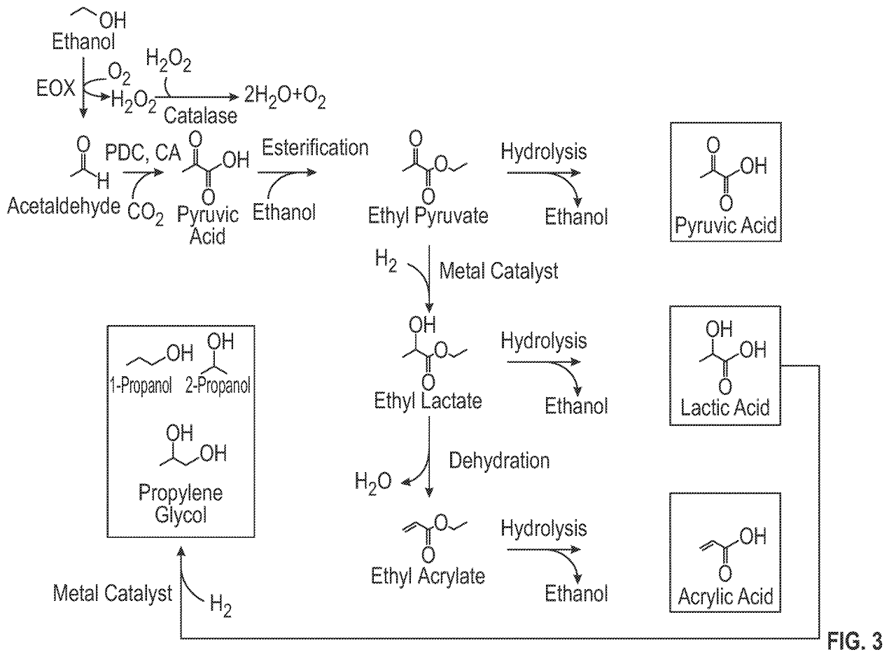

In an aspect, a method for the conversion of ethanol to a C3 compound is generally depicted in FIG. 3. Referring to FIG. 3, a method of the present disclosure comprises a Stage I where ethanol is contacted with an EOX. Stage 1 may further comprise contacting of acetaldehyde with a PDC in the presence of CO₂ or a source of CO₂ to generate pyruvic acid. Ethyl pyruvate may be generated through esterification of pyruvate in the presence of ethanol or an organic solvent. Esterification may be achieved using any suitable enzyme such as a CalB lipase (i.e., Novozymes 435 immobilized CalB). Because esterification using lipases typically requires anhydrous conditions, this reaction may occur in the presence of tert-butyl alcohol, methyl butyrate, or other solvent, potentially in a biphasic system.

Ethyl pyruvate may be converted to pyruvic acid by hydrolysis of the ethyl pyruvate, as shown in FIG. 3. The method may further comprise a Stage 2 which includes the reduction of ethyl pyruvate catalyzed by a hydrogenation catalyst to form ethyl lactate. Ethyl lactate may be converted to lactic acid by hydrolysis of the ethyl lactate as depicted in FIG. 3. In some aspects, the catalyst is chiral and catalyzes the production of predominately or exclusively R-lactic acid; alternatively, predominately or exclusively S-lactic acid.

In an alternative aspect, Stage 2 further comprises dehydration of ethyl lactate to form ethyl acrylate. Ethyl lactate may be converted to lactic acid by hydrolysis. In some aspects, the hydrolysis of ethyl lactate is catalyzed by a metal catalyst, alternatively a supported metal catalyst. The metal catalyst or supported metal catalyst may be the same as or different from the catalyst utilized to facilitate the reduction of pyruvate.

In another alternative aspect, Stage 2 comprises hydrogenation of lactic acid in the presence of a metal catalyst or supported metal catalyst and hydrogen to form a compound containing a propyl group. In an aspect, the compound containing a propyl group comprises 1-propanol, 2-propanol, propylene glycol or combinations thereof. The metal catalyst or supported metal catalyst may be the same as or different from the catalyst utilized to in an facilitate the hydrolysis reaction.

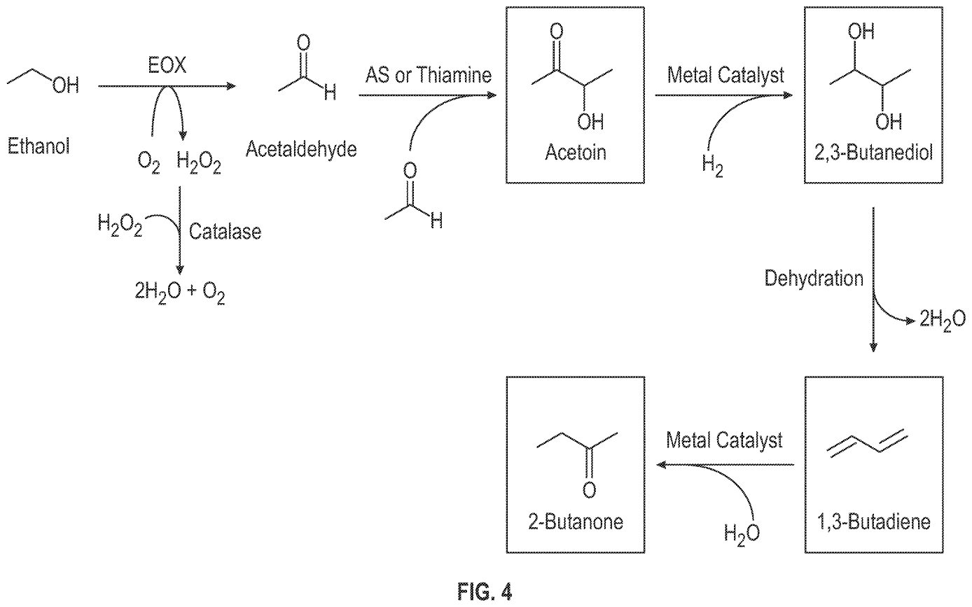

In an aspect, a method for the conversion of ethanol to a C4 compound is generally depicted in FIG. 4. Referring to FIG. 4, a Stage 1 of a method of the present disclosure comprises conversion of ethanol to acetoin. In an aspect, a method of the present disclosure comprises contacting of ethanol with an enzyme capable of catalyzing the selective aerobic oxidation of ethanol to acetaldehyde. In an aspect, this enzyme is an ethanol oxidase, EOX. Stage 1 may further comprise carboligation of two molecules of acetaldehyde to produce acetoin. Any suitable catalyst may be utilized to carry out the carboligation of acetaldehyde molecules. In an aspect, carboligation of the acetaldehyde is catalyzed by an acetoin synthase (AS). In an alternative aspect, carboligation of acetaldehyde is catalyzed by a thiamine-containing catalyst, carbene catalysts such as N-heterocyclic catalysts or combinations thereof. In some aspects, carboligation of acetaldehyde to produce acetoin is carried under conditions suitable to increase the stereospecificity of the reaction product; for example, the reaction product may comprise an excess of or exclusively a particular enantiomer, such as (R)-acetoin. In an alternative aspect, carboligation of acetaldehyde to produce acetoin is carried out under conditions suitable to produce a racemic product. In such aspects, carboligation is catalyzed by enzymes such as formolase (FLS) or PDC.

In an aspect, Stage 2 of the presently disclosed method comprises reduction of acetoin through hydrogenation over a metal catalyst to form 2,3-butanediol. In an aspect, butadiene may be formed by the dehydration of 2,3-butanediol. A method of the present disclosure may further comprise partial dehydration of 2,3-butanediol to form 2-butanone. Dehydration of 2,3-butanediol may be catalyzed by a metal catalyst that is the same as or different from the metal catalyst utilized to catalyze the hydrolysis of acetoin.

The methods of the present disclosure further comprise a pyruvate decarboxylase (PDC). Pyruvate decarboxylase (E.C. 4.1.1.1), also known as 2-oxo-acid decarboxylase, alpha-ketoacid decarboxylase, and pyruvic decarboxylase) is a homotetrameric enzyme that catalyzes the decarboxylation of pyruvic acid to produce acetaldehyde using magnesium and a thiamine pyrophosphate (TPP) cofactor. The enzyme is found in the cytoplasm of prokaryotes and cytoplasm and mitochondria of eukaryotes. In yeast, this enzyme is a key player in the fermentation process that produces ethanol. It is also present in some fish (including goldfish and carp) where it enables the animal to perform ethanol fermentation in low oxygen environments.

Once regarded as irreversible due to CO₂ evolution in the biologically-relevant reaction, decarboxylase reactions have proved reversible. In an aspect, a PDC suitable for use in the present disclosure may be sourced from microbes with high specific activity on pyruvate including those sourced from Acetobacter pasteurianus PDC, Zymobacter palmae PDC (ZpPDC), Zymomonas mobilis PDC (ZmPDC), and Saccharomyces cerevisiae PDC. In an aspect, the PDC has any of SEQ ID NO: 68 and SEQ ID NO:74. In an aspect, a PDC of the type disclosed herein, once reacted with acetaldehyde and carbon dioxide under suitable conditions forms pyruvate. In another aspect, a PDC of the type disclosed herein, once reacted with an aldehyde under suitable conditions forms acetoin.

In an aspect, the present disclosure comprises the conversion of acetaldehyde to acetoin via carboligation of two acetaldehyde molecules. Carboligation may be catalyzed by an acetoin synthase (AS). In an aspect, the AS is a pyruvate decarboxylase (PDC). Alternatively, the AS is a formolase. In yet another aspect, an AS suitable for use in the present disclosure is the E1 component of α-ketoglutarate dehydrogenase complex from Escherichia coli (EcSucA).

In an aspect, the AS is a formolase. Formolase (FLS) is a computationally designed enzyme developed by the laboratory of David Baker at the University of Washington, Seattle. Derived from benzaldehyde lyase (BAL), formolase contains the mutations mutant A28I, W89R, L90T, R188H, A394G, G419N, and A480W which allow it to catalyze the carboligation of three formaldehyde molecules to generate one molecule of dihydroxyacetone. The enzyme was also found to generate acetoin when fed acetaldehyde. In particular, FLS containing a L482S mutation, was found to improve activity through strengthening the contact between acetaldehyde and W480. The mutant enzyme exhibits a kcat of 0.63 s⁻¹ and a 72.95% improvement in K_{cat}/Kₘ versus the wild-type FLS. In an aspect, the FLS has any of SEQ ID NO.: 75 or SEQ ID NO.76.

In an aspect, the AS is the E1 component of an α-ketoglutarate dehydrogenase complexes. The E1 component of α-ketoglutarate dehydrogenase complexes (SucA) has been found to generate acetoin from acetaldehyde. The SucA enzyme of Vibrio vulnificus (VvSucA) was compared to an FLS for (R)-acetoin production. VvSucA showed the highest activity out of SucA proteins from E. coli (EcSucA), Mycobacterium bovis (MbSucA), Methylomicrobium alcaliphilum 20Z (MaSucA), and Methylomonas sp. DH-1 (MtSucA, MtputSucA). FLS and ZmPDC were found to produce 140% and 57% more acetoin than VvSucA, respectively. However, VvSucA showed high selectivity for R-acetoin formation (92% ee) while EcSucA and MbSucA showed an ee of 86 and 80%, respectively. FLS generated a racemic product while ZmPDC showed a preference for formation of the (S)-enantiomer. Thus, VvSucA or a homolog with similar properties may be useful if high enantioselectivity for (R)-acetoin production is required. The enantioselectivity of VvSucA could be improved through mutation (e.g. V298-H301 to GGG) to produce almost 100% (R)-acetoin, but only with a reduction of carboligating activity. However, a S324N mutant improved both stereoselectivity and activity of VvSucA while K228L and S324N/K228L mutants improved activity with less dramatic reductions in stereoselectivity compared with the glycine triple mutant. In an aspect, the SucA may have any of SEQ ID NO.: 77 through SEQ ID NO.: 82.

Generation of acetoin may also proceed using immobilized vitamin B1 or thiamine. In 2014, Lu et al. demonstrated that thiamine at 1 mol % with a 1.3 mol % amount of sodium bicarbonate could generate acetoin from acetaldehyde in sixteen hours at greater than 95% yield in water at 80° C.61 We postulate that thiamin immobilized to nanoparticles such as silica or maghemite-silica beads could be used in the EOR alongside the EOX enzyme to generate acetoin.62,63 Covalently bound B1-silica complexes are generated by adding a base such as triethylamine to a mixture of each component in methanol. Under these conditions, the O nucleophile of silica attacks the electrophilic C in the hydroxyethyl sidechain of thiamine, displacing a water molecule.

### Acetoin Hydrogenation to 2,3-Butanediol

To obtain 2,3-butanediol, acetoin generated from the enzymatic reactor will be hydrogenated over a ruthenium or other metal catalyst to generate the final product. Patents have been filed for this process in several countries, including one that details the hydrogenation of acetoin using a ruthenium or platinum catalyst with a selectivity higher than 90% (e.g, US20170327443, JP20155227298). 17,64 Stereoselective catalysts may be used to generate the meso, (R,R), or (S,S) compounds.

### Dehydration of 2,3-Butanediol to 1,3-Butadiene Via Metal Catalysis

2,3-butanediol dehydration to form 1,3-butadiene may be performed using a metal oxide, zirconia doped with alkaline-earth elements, or rare earth orthophosphate catalyst.65,66 A key element in ensuring high yield of butadiene is to limit pinacol rearrangement to yield butanone (methyl ethyl ketone, MEK) and isobutanol.

### Hydration of 1,3-Butadiene to 2-Butanone

The hydration of 1,3-butadiene to 2-butanone has been well characterized and is facilitated through use of a ruthenium catalyst. The catalyst may be synthesized from one equivalent of 1,10-phenathroline mixed with ruthenium trichloride.67

In an aspect, an EOX suitable for use in the present disclosure is an alcohol oxidase (AOX), a copper-radical oxidase such as a galactose oxidase (GAO), or a combination thereof.

In an aspect, the EOX is an alcohol oxidase (E.C. 1.1.3.13). AOX is a ubiquitous flavin-dependent enzyme that oxidizes lower primary alcohols to aldehydes using oxygen as a terminal oxidant. The AOX may be sourced from methylotrophic yeasts or methylotrophic yeast waste such as members of the genus Candida, Hansenula, Kloeckera, Torulopsis, Candida, Pichia, Hansenula, Hanseniaspora, and Metschnikowia. Alternatively, AOX enzymes used in this process may be sourced from methanol-utilizing bacteria like Methylococcus capsulatus, thermophilic soil fungi such as Thermoascus aurantiacus, brown rot fungus such as Gloeophyllum trabeum or the white-rot basidiomycete Phanerochaete chrysosporium.

Methylotrophic yeast waste generated in fermentative processes is an attractive source of AOX. These organisms include, but are not limited to, members of the genera Kloeckera, Torulopsis, Candida, Pichia, Hansenula, Hanseniaspora, and Metschnikowia. Methylotrophic yeasts are widely employed in fermentative processes for protein production and chemical synthesis. In many cases, these yeasts are used to generate proteins heterologously under control of the methanol-inducible AOX1 promoter. The endogenous AOX1 gene can be retained (Mut⁺ strains), deleted (Muts), or deleted along with that of the minor alcohol oxidase AOX2 (Mut⁻). Generally, higher protein titers are achieved in strains capable of utilizing methanol as a carbon source, while AOX genes may be deleted to improve protein titers in non-methanol induced processes. In an aspect, the AOX is sourced from Mut⁺ cells generated as a byproduct of methylotrophic yeast fermentation. Cell density in these processes can reach a final level of from about 350 g/L to about 450 g/L wet cells. When grown in methanol, AOX can comprise 30% of soluble cellular protein, 20% of cell-free extracts and 80% of cell volume. Alternatively, AOX sequences used in this process may be sourced from organisms other than methylotrophic yeasts.

An AOX suitable for use in the present disclosure may be sourced from any suitable organism, nonlimiting examples of which are presented in Table 1.

TABLE 1

| Organism | Genbank ID |
|---|---|
| Achatina achatina | NA |
| Achatina fulica | NA |
| Arion ater | NA |
| Aspergillus ochraceus | NA |
| Aspergillus ochraceus AIU 031 | |
| Aspergillus nidulans | 40747510 |
| Aspergillus terreus | GFF15729.1 |
| Aspergillus terreus MTCC 6324 | GFF15192.1 |
| | GFF19169.1 |
| | EAU30325.1 |
| | XP_001217810.1 |
| | AFP17823.1 |
| Basidiomycota | NA |
| Basidiomycota B191039 | |
| Byssochlamys spectabilis | NA |
| Byssochlamys spectabilis RI017 | |
| Paecilomyces variotii | |
| Candida boidinii | AAV66467.2 |
| Candida methanolica | Q00922.1 |
| Candida koshuensis | AAA34321.1 |
| Candida olivarium | |
| Candida ooitensis | |
| Candida queretana | |
| Candida silvicola | |
| HanensulaHansenula alcoolica | |
| Kloeckera boidinii | |
| Torulopsis enokii | |
| Candida cariosilignicola | BAF63435.1 |
| Candida guilliermondii | NA |
| Pichia guilliermondii | |
| Yamadazyma guilliermondii | |
| Endomyces guilliermondii | |
| Candida methanolovescens | BAE94372.1 |
| Ogatea minuta | |
| Candida methanosorbosa | BAF63437.1 |
| Candida methanosorbosa M- | |
| 2003 | |
| Candida sithepensis | NA |
| Candida sonorensis | BAF63431.1 |
| Torulopsis sonorensis | |
| Candida sp. (in: | |
| Saccharomycetales) | |
| Candida sp. (in: | |
| Saccharomycetales) 25-A | |
| Candida succiphila | BAF63438.1 |
| Candida tropicalis | AAS46880.1 |
| | AAS46879.1 |
| | AAS46878.1 |
| | CAB75353.1 |
| | ACX81419.1 |
| Comamonas sp. | WP_034404478.1 |
| Comamonas sp. UVS | WP_003050665.1 |
| | WP_057094210.1 |
| | WP_046461751.1 |
| | WP_003068550.1 |
| | WP_003059940.1 |
| | WP_003065255.1 |
| Gloeophyllum trabeum | ABI14440.1 |
| Hansenula polymorpha | CAM84032.1 |
| Ogataea polymorpha | CAM84031.1 |
| Pichia angusta | CAM84030.1 |
| HanensulaHansenula angusta | P04841.1 |
| Ogataea angusta | ESW98254.1 |
| Ogataea angusta DL-1 | XP_013934137.1 |
| Ogataea angusta NCYC 495 | |
| Helix aspersa | NA |
| Kuraishia capsulata | BAF63439 |
| Lachnellula arida | TVY19911.1 |
| Lachnellula cervina | TVY50643.1 |
| Lachnellula occidentalis | TVY35490.1 |
| Lachnellula subtilissima | TVY31638.1 |
| Lachnellula suecica | TVY82639.1 |
| | TVY82378.1 |
| Lachnellula willkommii | TVY85881.1 |
| Methylococcus capsulatus | WP_017366030.1 |
| | WP_010959550.1 |
| | WP_017365658.1 |
| | WP_041361040.1 |
| Methylophilus methylotrophus | NA |
| Ochrobactrum sp. | WP_007878006.1 |
| Ochrobactrum sp. AIU 033 | WP_121985034.1 |
| | WP_121981009.1 |
| | WP_109369391.1 |
| | WP_109367817.1 |
| | WP_105707908.1 |
| | WP_105535410.1 |
| | WP_105535243.1 |
| | WP_105519965.1 |
| | WP_100558784.1 |
| | WP_100557726.1 |
| | WP_094543178.1 |
| | WP_094513902.1 |
| | WP_063620893.1 WP_029928001.1 |
| | WP_029929015.1 |
| | WP_024899612.1 |
| | WP_021586864.1 |
| | WP_010660975.1 |
| | WP_010659752.1 |
| Ogataea glucozyma | BAF63441.1 |
| Ogataea henricii | BAF63442.1 |
| Ogataea methanolica | AAF02495.1 |
| Pichia pinus | AAF02494.1 |
| Ogataea minuta | BAE94372.1 |
| Ogataea naganishii | BAF63444.1 |
| Ogataea philodendri | BAF63445.1 |
| Ogataea pignaliae | BAF63434.1 |
| | BAF63433.1 |
| Ogataea pini | AAQ99151.1 |
| Ogataea siamensis | NA |
| Ogataea trehalophila | BAF63446.1 |
| Ogataea wickerhamii | BAF63440.1 |
| Passalora fulva | AAF82788.1 |
| Penicillium chrysogenum | KZN85194.1 |
| | AAL56054.1 |
| | XP_002557820.1 |
| | CAP80622.1 |
| Penicillium purpurascens | NA |
| Penicillium purpurascens AIU | |
| 063 | |
| Phanerochaete chrysosporium | CDG66232.1 |
| Phanerochaete chrysosporium | |
| DSMZ 1547 | |
| Phanerochaete chrysosporium K- | |
| 3 | |
| Phlebiopsis gigantea | NA |
| Pichia pastoris | AAB57850.1 |
| Komagataella pastoris | AAB57849.1 |
| Komagataella phaffii | CCA41016.1 |
| Komagataella pseudopastoris | CCA40305.1 |
| Endomyces pastoris | XP_002494271.1 |
| Petasospora pastoris | CAY72092.1 |
| Zygosaccharomyces pastoris | F2QY27.1 |
| Zygowillia pastoris | |
| Zymopichia pastoris | |
| Komagataella pastoris GS115 | |
| Komagataella pastoris IFP 206 | |
| Komagataella pastoris X33 | |
| Pichia putida | NA |
| Polyporus obtusus | NA |
| Poria contigua | NA |
| Radulodon casearius | NA |
| Thodotorula toruloides | XP_016273069.1 |
| | EMS21950.1 |
| | XP_016271944.1 |
| | EMS20825.1 |
| Thermoascus aurantiacus | NA |
| Thermoascus aurantiacus NBRC | |
| 31693 | |
| Trametes cinnabarina | CDO76344.1 |

In an aspect, the enzyme is an inherently stable form of an AOX from thermophilic organisms such as Candida methanosorbosa (Tₒₚₜ=45° C.), Ogataea thermomethanolica (Tₒₚₜ=50° C.), or Phanerochaete chrysosporium (Tₒₚₜ=50° C.).

In an aspect, the EOX is a member of the copper radical oxidase family. For example, and without limitation, a copper radical oxidase suitable for use in the present disclosure is galactose oxidase (GAO, EC 1.1. 3.9). GAO is one of the most extensively studied alcohol oxidases with respect to both mechanistic investigations and also practical applications. Other members in the copper radical oxidase family may be suitable as an EOX, but may not necessarily catalyze the oxidation of galactose. Wild-type GAO is highly selective for D-galactose and D-talose but will not oxidize other sugars (e.g., D-glucose, D-mannose) as shown in Reaction 1.

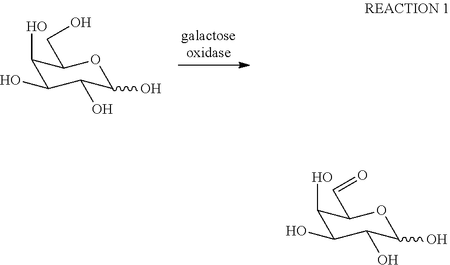 [SMILES: OCC1OC(O)C(O)C(O)C1O ; O=CC1OC(O)C(O)C(O)C1O]

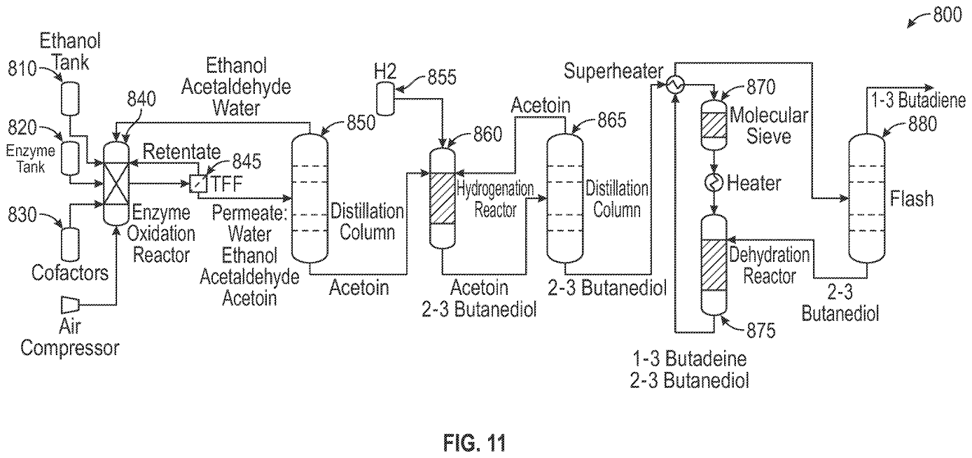

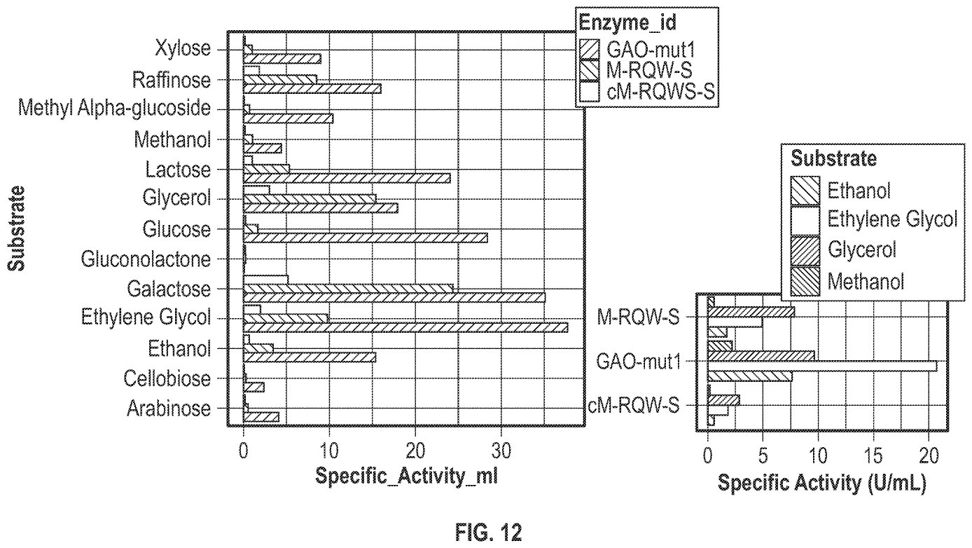

GAO is a copper-dependent alcohol oxidase that oxidizes galactose residues either as monosaccharides or glycoconjugates that contain galactose at the nonreducing end. GAO forms a novel metalloradical complex, comprised of a protein radical coordinated to a copper ion in the active site. The unusually stable protein radical is formed from the redox-active side chain of a cross-linked tyrosine residue (Tyr-Cys). In an aspect, the AOX (e.g., GAO) has any of SEQ ID NO: 1 through SEQ ID NO:67. In an aspect, the GAO is a mutated Fusarium graminearum GAO (FgGAO Mut-1) capable of oxidizing ethanol. FgGAO Mut-1 was found to oxidize ethanol with a specific activity of about 8 U mL⁻¹ of bacterial lysate, see FIG. 11.

In an aspect, any of the enzymes disclosed herein is a wild type enzyme, a functional fragment thereof or a functional variant thereof. “Fragment” as used herein is meant to include any amino acid sequence shorter than the full-length enzyme (e.g., AOX, GAO), but where the fragment maintains a catalytic activity sufficient to meet some user or process goal. Fragments may include a single contiguous sequence identical to a portion of the enzyme sequence. Alternatively, the fragment may have or include several different shorter segments where each segment is identical in amino acid sequence to a different portion of the amino acid sequence of the enzyme but linked via amino acids differing in sequence from the wildtype enzyme. Herein, a “functional variant” of the enzyme refers to a polypeptide which has at one or more positions of an amino acid insertion, deletion, or substitution, either conservative or non-conservative, and wherein each of these types of changes may occur alone, or in combination with one or more of the others, one or more times in a given sequence but retains catalytic activity.

In the alternative or in combination with the aforementioned mutations, the enzyme may be mutated to improve the catalytic activity. Mutations may be carried out to enhance the activity of the protein or a homolog, increase the protein stability in the presence of acetaldehyde and/or hydrogen peroxide, and increase protein yield. In some aspects, a variant of an enzyme suitable for use in the present disclosure comprises at least 50% sequence identity with any sequence disclosed herein, or at least 60%, or at least 70%, or at least 75%, or at least 80%, or at least 85%, or at least 90%, or at least 95% or alternatively from about 50% to about 95%.

Herein, reference has been made to “sources” of enzymes. It is to be understood this refers to the biomolecule as expressed by the named organism. It is contemplated the enzyme may be obtained from the organism or a version of said enzyme (wildtype or recombinant) provided as a suitable construct to an appropriate expression system. In an aspect, any enzyme of the type disclosed herein may be cloned into an appropriate expression vector and used to transform cells of an expression system such as E. coli, Saccharomyces, Pichia pastoris, Aspergillus sp., Myceliophthora sp., or Trichoderma sp.

A “vector” is a replicon, such as plasmid, phage, viral construct or cosmid, to which another DNA segment may be attached. Vectors are used to transduce and express a DNA segment in cells. As used herein, the terms “vector” and “construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more AOX gene expression cassettes may be or are ligated. Herein, a cell has been “transformed” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell.

In an aspect, the reactions disclosed herein may further include one or more purified enzyme cofactors. Nonlimiting examples of purified enzyme cofactors suitable for use in the present disclosure include thiamine pyrophosphate, NAD+, NADP+, pyridoxal phosphate, methyl cobalamin, cobalamine, biotin, Coenzyme A, tetrahydrofolic acid, menaquinone, ascorbic acid, flavin mononucleotide, flavin adenine dinucleotide, and Coenzyme F420. Such cofactors may be included in the enzyme preparation and/or be added at various points during the reaction. In some aspects, cofactors included with the enzyme preparation may be readily regenerated with oxygen and/or may remain stable throughout the lifetime of the enzyme(s).

As will be understood by one of ordinary skill in the art with the benefit of the present disclosure, reactions of the type disclosed herein (e.g., enzyme oxidation of ethanol) may result in the production of byproducts (e.g., hydrogen peroxide) that can detrimentally impact other components of the reaction mixture. For example, hydrogen peroxide may degrade the enzyme resulting in a loss of catalytic activity. In such aspects, mitigation of the detrimental effects of hydrogen peroxide may be carried out such as by the introduction of a catalase (E.C. 1.11.1.61), the use of a hydrogen peroxide-resistant enzyme or combinations thereof.

In an aspect of the present disclosure a metal catalyst, alternatively a supported metal catalyst is employed to facilitate production of the disclosed C3 compounds. Specifically, pyruvic acid hydrogenation to lactic acid and dehydration to acrylic acid via is facilitated by a metal catalyst of the type disclosed herein. For example, to obtain lactic acid, pyruvate generated from the enzymatic reactor will be hydrogenated over a metal catalyst (e.g., platinum, copper, a ruthenium-containing catalyst) to first generate a lactate intermediate and then the final product. This can be done non-asymmetrically to produce a lactic acid racemate or asymmetrically to produce primarily D-lactate or L-lactate.

Lactic acid dehydration to form acrylic acid may be performed in the metal catalyst reactor simultaneously with the hydrogenation step. This reaction may generate side products resulting from condensation, decarboxylation, and esterification.

Additionally, to obtain propylene glycol and n-propanol, pyruvate generated from the enzymatic reactor may be hydrogenated over a metal catalyst (e.g., a ruthenium-containing catalyst) to first generate a lactate intermediate and subsequently the final product.

In an aspect, the hydrogenation catalyst comprises a supported-metal catalyst such a heterogenous metal catalyst (HMC). In an aspect, the support comprises carbon, silica, alumina, titania (TiO₂), zirconia (ZrO₂), a zeolite, or any combination thereof, which contains less than about 1 weight percent (wt. %), alternatively less than about 0.1 wt. % or alternatively less than about 0.01 wt. % SiO₂ binders based on the total weight of the support.

Suitable support materials are predominantly mesoporous or macroporous, and substantially free from micropores. For example, the support may comprise less than about 20% micropores. In an aspect, the support of the HMC is a porous nanoparticle support. As used herein, the term “micropore” refers to pores with diameter <2 nm, as measured by nitrogen adsorption and mercury porosimetry methods and as defined by IUPAC. As used herein, the term “mesopore” refers to pores with diameter from ca. 2 nm to ca. 50 nm, as measured by nitrogen adsorption and mercury porosimetry methods and as defined by IUPAC. As used herein, the term “macropore” refers to pores with diameters larger than 50 nm, as measured by nitrogen adsorption and mercury porosimetry methods and as defined by IUPAC.

In an aspect, the HMC support comprises a mesoporous carbon extrudate having a mean pore diameter ranging from about 10 nm to about 100 nm and a surface area greater than about 20 m² g⁻¹ but less than about 300 m² g⁻¹ Supports suitable for use in the present disclosure may have any suitable shape. For example, the support may be shaped into 0.8-3 mm trilobes, quadralobes, or pellet extrudates. Such shaped supports enable the used of fixed trickle bed reactors to perform the final oxidation step under continuous flow.

In an aspect, the HMC comprises gold, Au. In one or more aspects, the metal comprises a Group 8 metal (e.g., Re, Os, Ir, Pt, Ru, Rh, Pd, Ag), 3d transition metals, early transition metals, or combinations thereof. In an alternative aspect, a dehydration catalyst comprises hafnium, tantalum, zinc or combination thereof on a support such as a zeolite or a β-zeolite.

The HMC may be prepared using any suitable methodology. For example, the HMC may be prepared using gas phase reduction of the support (e.g., carbon) impregnated with metal salts in hydrogen at temperatures ranging from greater than about 200° C. to about 600° C. In an alternative aspect, the HMC may be prepared using liquid phase reduction of the support impregnated with metal salts immersed in an aqueous oxygenate (e.g. formate, gluconate, citrate, ethylene glycol) solution at temperature between about 0° C. and about 100° C. Alternatively, the impregnated support can be loaded into the hydrogenation reactor in a non-reduced form and reduced on stream by the reactants of the process during startup. Liquid Phase Reduction (LPR) is a synthetic method to obtain a core-shell dispersion of the active metallurgy over a surface annulus of the extrudate.

These materials can be prepared via incipient wetness or bulk adsorption of a metal precursor salt solution onto the extrudate support followed by either Gas Phase Reduction (GPR) at temperatures between 100° C. and 500° C. under an H₂/N₂ atmosphere or followed by LPR using an alkaline aqueous formate salt solution between 20° C. and 100° C.

### Reactor Configurations

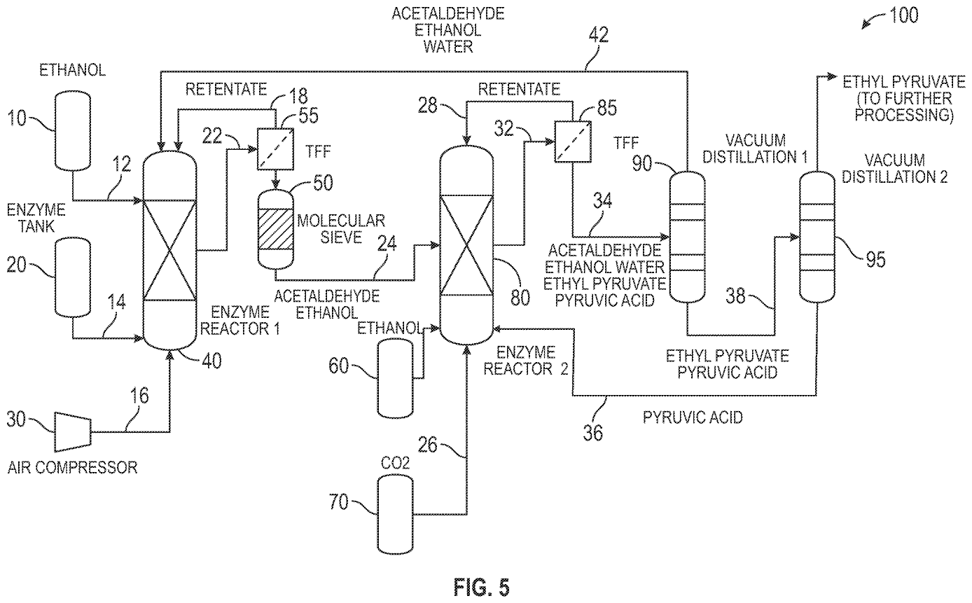

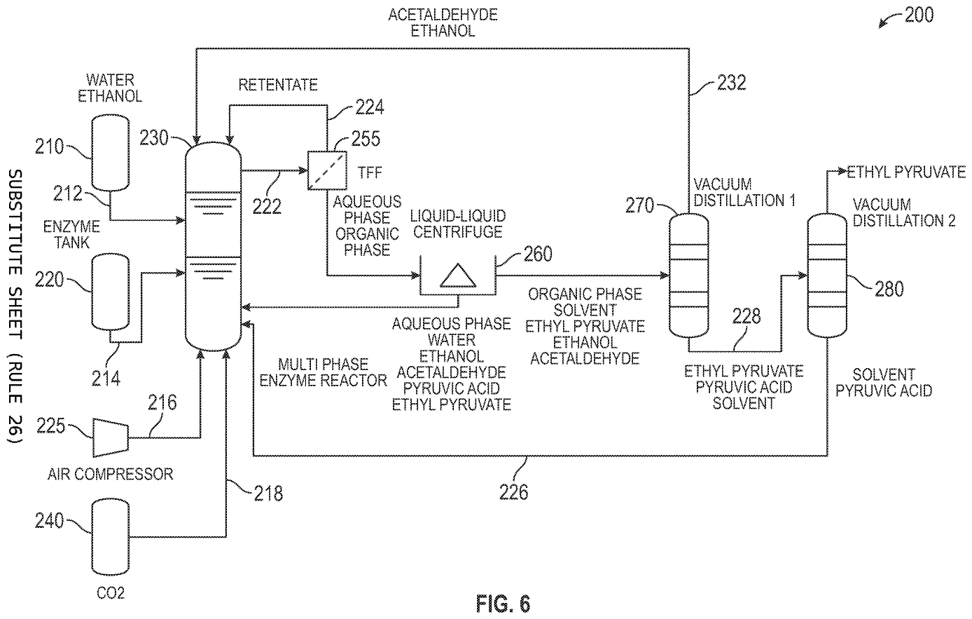

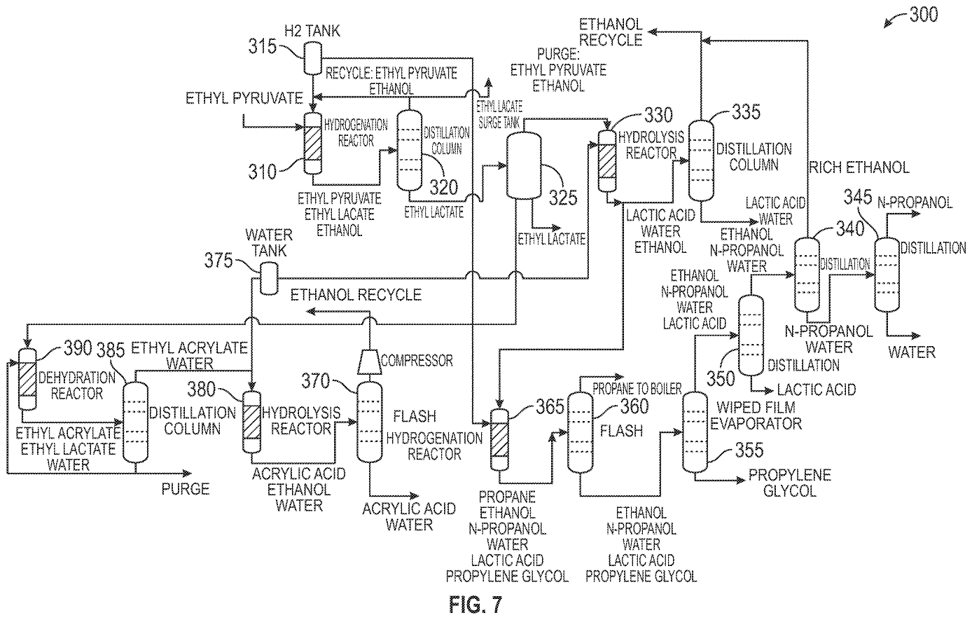

In an aspect, the present disclosure contemplates novel reactor configurations for carrying out reactions of the present disclosure. FIGS. 5 and 6 contemplate the production of ethyl pyruvate and/or pyruvic acid as the final product while FIG. 7 contemplates the conversion of ethyl pyruvate to different end-products. Referring to FIG. 5, an aspect of a system for production of ethyl pyruvate 100 is depicted. Reactants ethanol, enzyme and oxygen are introduced to the system 100 from the ethanol-containing tank 10 via conduit 12; the enzyme tank 20 via conduit 14 and the air compressor 30 via conduit 16. Conduits 12, 14 and 16 feed into Enzyme Reactor 1, 40. Enzymatic Reactor 1, 40, could be a sparged bubble column (as shown), an airlift column, a stirred sparged bioreactor, or a falling film high-pressure oxidation vessel. The enzyme reactor, 40, may operate at high conversion (potentially as multiple reactors in series) with minimal water concentration. In an aspect, operating ranges for the Enzyme Reactor 1,40, are from about 20° C. to about 60° C. at pressures ranging from about 1 bar to about 15 bar. In an aspect, in the Enzyme Reactor 1, 40, ethanol is oxidized to acetaldehyde by an EOX of the type disclosed herein (e.g., AOX, GAO), while the hydrogen peroxide generated is reacted away to oxygen and water by catalase. Enzyme reactor effluent may be sent to a tangential flow filter (TFF) 55 via conduit 22 to preserve enzymes in the enzyme reactor as recycled retentate via conduit 18, with pyruvic acid permeate flowing further down the process.

Enzyme free effluent may be sent from Enzyme Reactor 1, 40, through a molecular sieve 50 in order to dewater the effluent, which is then sent to Enzyme Reactor 2 80 via conduit 24. Additional ethanol and CO₂ may be introduced to Enzyme Reactor 2, 80, from an ethanol-containing tank 60 via conduit 46 and a carbon-dioxide containing tank 70 via conduit 26. This enzymatic reactor (i.e., Enzyme Reactor 2) 80 could be a sparged bubble column (as shown), an air lift column, a stirred sparged bioreactor, or a falling film high pressure oxidation vessel. Enzyme Reactor 2, 80, may be sparged with carbon dioxide, either as a high-pressure gas or with supercritical CO₂, and is fed liquid ethanol. In Enzyme Reactor 2, 80, acetaldehyde is first carboxylated to pyruvic acid, which is then rapidly esterified with ethanol to ethyl pyruvate. In an aspect, water concentration is kept low to prevent the back reaction of ethyl pyruvate and water to pyruvic acid and ethanol. Enzyme reactor effluent may be sent to a TFF 85 via conduit 32 to preserve enzymes in the enzyme reactor as recycled retentate via conduit 28, with pyruvic acid permeate flowing further down the process.

Enzyme Reactor 2, 80, effluent may first be sent to a Vacuum Distillation Column 1 90 via conduit 34, while acetaldehyde, ethanol, and water are distilled and recycled back to Enzyme Reactor 1, 40, via conduit 42. Bottoms, which contain ethyl pyruvate and unreacted pyruvic acid may then passed to Vacuum Distillation Column 2, 95, via conduit 38. Pyruvic acid, with a boiling point of 165° C., may be removed as bottoms via conduit 36 and returned to Enzyme Reactor 2,80, while ethyl pyruvate, with a boiling point of 144° C., is removed as distillate, which can be the end-product or passed on for further processing.

In an alternative aspect, a process flow diagram for the chemoenzymatic production of C3 compounds from ethanol is depicted in FIG. 6. Referring to FIG. 6, reactants ethanol, oxygen, carbon dioxide and enzyme are introduced to the system 200 from the ethanol-containing tank 210 via conduit 212; the enzyme tank 220 via conduit 214, the air compressor 225 via conduit 216 and the carbon dioxide tank 240 via conduit 218. Conduits 212, 214, 216 and 218 feed into Multi-Phase Enzyme Reactor (MPER) 1, 230. Carbon dioxide may be introduced to the MPER as a gas, liquid, or supercritical fluid.

Herein a multiphase reactor refers to any vessel or region in space where more than one phase (gas, liquid, or solid) come into contact and result in a chemical change. Multiphase reactors provide an environment conducive for a chemical reaction: specifically, temperature and pressure conditions, and flow regimes, flow regime transitions, local and global fluid dynamics parameters, and mass transfer conditions that facilitate reaction catalysis. The MPER 230 disclosed herein is a novel 5-phase reactor comprising a gas phase, an aqueous liquid phase, an organic liquid phase, a hydrophilic solid phase, and a hydrophobic solid phase. In an aspect, a water-immiscible organic solvent (e.g., heptanol) is also present in the MPER 230, and is recycled throughout the process.

In an aspect, enzymes are added to the MPER 230 on immobilized supports, with EOX, catalase, and PDC on hydrophilic supports, in order to maintain their activity in the aqueous phase, and CalB on a hydrophobic support, in order to maintain its activity in the organic phase. The MPER 230 of the present disclosure is designed such that the sparging action of air and CO₂ vigorously mixes the reactor, ensuring continuous intimate contact and mass transfer between the aqueous and organic phases.

In an aspect, multiple species are present in the MPER 230. The partition coefficient, or Log P, of a species is indicative of the physicochemical preference for aqueous or organic phases. A Log P of <0 indicates a preference for aqueous, while a Log P>0 indicates a preference for organic. The Log P of various species disclosed herein is presented in Table 2.

TABLE 2

| Species | LogP | Prefers Phase: |
|---|---|---|
| Ethanol | −0.18 | Both |
| Acetaldehyde | −0.34 | Aq |
| Pyruvic Acid | −0.3 | Aq |
| Ethyl Pyruvate | 0.4 | Org |
| Pyruvate ion | <<0 | Aq |

All species will be present to some extent in both phases, but ethyl pyruvate has a marked preference for the organic phase. This is desirable, as it ensures that no water will be present to back-react ethyl pyruvate to pyruvic acid. Additionally, only pyruvic acid and not pyruvate will partition into the organic phase, where it is reacted away. This acts as a “sink” for the pyruvic acid present in the aqueous phase, pulling equilibrium away from pyruvate ions, which helps prevent excessive acidification in the system. Additionally, the MPER can operate with long residence times to minimize the pretense of the pyruvate intermediate to further minimize acidification.

This MPER design also enables the use of extensive buffering in the aqueous phase to control pH without having to separate out the buffering salts. pH control is a factor in optimizing the enzyme activity, stability, and spacetime yield. Common buffering agents include cations paired with anions. Nonlimiting examples of common cations being ammonium, sodium, potassium, and calcium; while nonlimiting examples of common anions being phosphate, sulfate, citrate, and gluconate.

Referring to FIG. 6, MPER 230 reactor effluent may be sent to a TFF 255 via conduit 222. Since enzymes are retained on supports, the TFF may comprise a microfilter, which will enable both aqueous and organic phases to pass through, while retaining the immobilized enzymes in the MPER 230.

Reactor effluent may then be sent to a liquid-liquid centrifuge 260 via conduit 242 to split the aqueous and organic phases. In an aspect, aqueous phase is recycled back to the MPER 230 via conduit 244, while the organic phase is passed through a series of vacuum distillation columns 270 and 280 via conduit 228. The first vacuum distillation column 270 may function to distill off ethanol and acetaldehyde (along with any entrained water) which can be recycled to MPER 230 via conduit 232. In an aspect, the second vacuum distillation column 280 serves to separate ethyl pyruvate as distillate from the solvent and residual pyruvic acid. Residual pyruvic acid can be recycled to the MPER 230 via conduit 226.

In an aspect, ethyl pyruvate generated utilizing either system 100 or system 200 can be subjected to further processing as depicted in FIG. 7. With reference to FIG. 7, exemplary reactions for the conversion of ethyl pyruvate include introduction of ethyl pyruvate and hydrogen 315 to a hydrogenation reactor 310 the products of which including ethyl lactate can be conveyed to a distillation column 320. Excess ethyl lactate may be stored in a surge tank 325 prior to conveyance to a hydrolysis reactor 330 where it can be hydrolyzed and subsequently subjected to a distillation column 335 to obtain purified lactic acid. Ethyl lactate may be dehydrated in a dehydration reactor 390 to produce ethyl acrylate, unreacted ethyl lactate and water as a product mixture that can be conveyed to a distillation column 385 for further purification. In other aspects, ethyl lactate is conveyed to a hydrolysis reactor 380 and subsequently to flash distillation 370 to obtain purified acrylic acid. In yet other aspects, ethyl lactate is conveyed to a hydrogenation reactor to generate propanol, ethanol, n-propanol, water, lactic acid and propylene glycol as a product mixture. Isolation of the component mixtures may be affected by an appropriate distillation column, e.g., 345, 350, 355 and 360.

In some aspects, once ethyl pyruvate has been produced and separated, it can be utilized as an end product, or it can be hydrogenated over a metal catalyst to ethyl lactate. In an aspect, this reaction takes place in a trickle bed reactor, with hydrogen introduced into the reactor feed line as hydrogenation feedstock. Though not shown, temperature and pressure into the hydrogenation reactor will be controlled via feed pumps, compressors, and heat exchangers. The hydrogenation reactor can be operated in a downward gas-liquid trickle bed flow where the extrudates are on the order of about 1 mm to about 10 mm in primary length. The extrudates can be pellets, trilobes, quadrilobes, tablets, or wagon wheels. In an aspect, the extrudate is a trilobe. The extrudates are fixed in place while the gas channels through the interparticle void space and the liquid flows as rivulets over the external support surface. Alternatively, the primary length of the extrudates can be on the order of from about 0.01 mm to about 1 mm. In an aspect, the catalyst is spherical and neutrally buoyant. With this catalyst, the hydrogenation reactor can be operated in slurry flow either in a plug flow regime where the liquid, gas bubbles, and catalyst are moving uniformly in a single direction. Alternatively, the reactor can be operated in slurry flow in a well-mixed regiment via a mechanical agitator or using the gas to ebullate the slurry. In an aspect, the reactor is a downward trickle bed. A mixture of ethanol, ethyl lactate, and ethyl pyruvate flow on to a distillation unit.

This next distillation unit will likely need to be a distillation column, as ethyl lactate is fairly near-boiling with ethyl pyruvate (154° C. vs 145° C.). A vacuum distillation column is shown in the PFD to achieve high purity lactic acid bottoms. The column can operate at atmospheric pressures, but ideally it operates at 2-8 psia and <80° C. to preserve product integrity.

Purified ethyl lactate can now either be hydrolyzed to produce lactic acid (a potential end-product), and from there propylene glycol; or ethyl lactate can be dehydrated to produce ethyl acrylate and from there acrylic acid. For lactic acid, ethyl lactate may first be passed through a hydrolysis reactor, along with additional feed water. In an aspect, this reaction is catalyzed by the addition of a strong acid, alternatively catalyzed via a strong acid resin at moderate (80° C.) temperatures. In an aspect, the hydrogenation reactor employs pervaporation or reactive distillation to actively remove ethanol and promote further hydrolysis. Alternatively, a sulfonated zirconia catalyst bed at moderate (e.g., 80° C.) temperature could be employed as a heterogeneous hydrolysis catalyst.

In an aspect, lactic acid either undergoes distillation to produce high purity lactic acid end-product as bottoms, or undergoes hydrogenation to produce propylene glycol. In another aspect, propylene glycol is produced by the hydrogenation of lactic acid over a metal catalyst. In such aspects, hydrogenation with the metal catalyst occurs in a trickle bed reactor, with hydrogen introduced into the reactor feed line as hydrogenation feedstock. Though not shown, temperature and pressure into the hydrogenation reactor will be controlled via feed pumps, compressors, and heat exchangers. The hydrogenation reactor can be operated in a downward gas-liquid trickle bed flow where the extrudates are on the order of from about 1 mm to about 10 mm in primary length. The extrudates can be pellets, trilobes, quadrilobes, tablets, or wagon wheels. In an aspect, the extrudate is a trilobes. Extrudate may be fixed in place while the gas channels through the interparticle void space and the liquid flows as rivulets over the external support surface. Alternatively, the primary length of the extrudates can be on the order of from about 0.01 mm to about 1 mm. In an aspect, the extrudates are spherical and neutrally buoyant. With this catalyst, the hydrogenation reactor can be operated in slurry flow either in a plug flow regime where the liquid, gas bubbles, and catalyst are moving uniformly in a single direction. Alternatively, the reactor can be operated in slurry flow in a well-mixed regiment via a mechanical agitator or using the gas to ebullate the slurry. In an aspect, the reactor is a downward trickle bed reactor. A mixture of propane, ethanol, n-propanol, water, lactic acid, and propylene glycol exits the reactor.

In an aspect, the complex mixture is subjected to a multi-step distillation process. The first distillation can be a low temperature single stage flash and serves to separate propane from the rest of the materials. Propane is substantially more volatile, so it should separate as distillate. Propane can then be feed to a boiler. Ethanol, n-propanol, water, lactic acid, and propylene glycol exit the flash as bottoms to the next distillation.

Propylene glycol is significantly less volatile than the other species, with a normal boiling point of 188° C., versus that of the next least volatile species, lactic acid at 122° C. As propylene glycol is quite viscous, a wiped film evaporator would be the ideal distillation unit, with ethanol, n-propanol, water, and lactic acid distillate recycled to enhance overall yield (with some going to a purge), and propylene glycol taken as bottoms. For product integrity purposes, this will likely be a vacuum distillation.

Distillate from the wiped film evaporator contains (in order of less to more volatile) lactic acid, water, n-propanol, and ethanol, all of which are fairly close-boiling. In an aspect, this is carried out utilizing a 3-distillation column train, where lactic acid is purified as bottoms in the first column, ethanol is purified as distillate in the second column, and n-propanol is recovered as distillate in the third column. Both ethanol and n-propanol form azeotropes with water, so vacuum or extractive distillation may need to be employed, depending on purity requirements.

To produce acrylic acid, ethyl lactate is first passed through a dehydration reactor to produce ethyl acrylate. Vapor entering the reactor must first be heated to greater than about 300° C. The dehydration reactor may be packed with a tantalum beta zeolite, although hafnium beta zeolites, tin beta zeolites, or a HfZn catalyst could also be utilized. The reactor may be operated at low conversions (10-20%).

The dehydration reactor effluent contains ethyl lactate, ethyl acrylate, and water. Ethyl acrylate and water both have normal boiling points of 100° C., and as such will partition to distillate, while ethyl lactate has a normal boiling point of 154° C., and will be found in the bottoms, likely with some water. Depending on ethyl acrylate purity requirements, this distillation could be a single stage flash (i.e. flash drum, MVR, etc.) or a distillation column. For product integrity purposes, this will likely be a vacuum distillation.

Ethyl acrylate distillate may then be sent to a hydrolysis reactor to produce acrylic acid, along with additional feed water. This reaction could be catalyzed by the addition of a strong acid, or via a strong acid resin at moderate (80° C.) temperatures. This hydrogenation reactor could potentially employ pervaporation or reactive distillation to actively remove ethanol and promote further hydrolysis. Alternatively, a sulfonated zirconia catalyst bed at moderate (80° C.) temperature could be employed as a heterogeneous hydrolysis catalyst. The reactor effluent contains acrylic acid, water, and ethanol.

The acrylic acid in the reactor effluent can be readily purified from water and ethanol by distillation. Acrylic acid has a normal boiling point of 141° C., and will partition to the bottoms. As acrylic acid is highly self-reactive, this separation may be carried out under vacuum to reduce the temperature and maintain product integrity. Depending on purity requirements, this distillation can take place in a single stage flash (i.e. flash drum, mechanical vapor recompressor, etc.), or a distillation column.

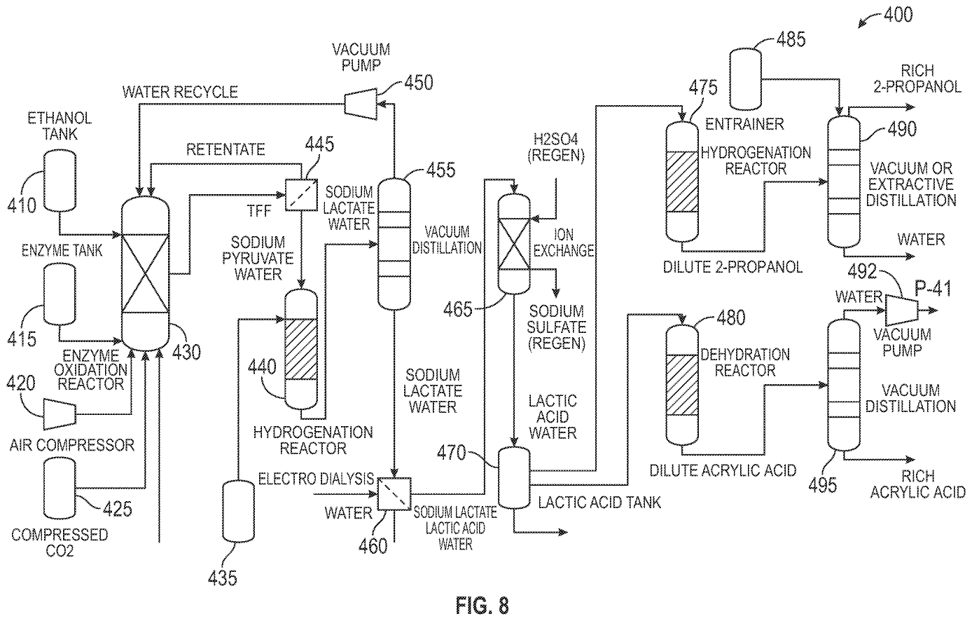

FIG. 8 depicts an alternative reactor process 400 for production of HVCs. With reference to FIG. 8, the reactants ethanol, enzyme and carbon dioxide can be introduced to an enzyme reactor 430 from storage tanks 410, 415 and 425, respectively. The flow of these materials within the enzyme reactor 430 can be added by a compressor 420. The sodium pyruvate and water products exiting enzyme reactor 430 can be passed through a TFF 445 to a hydrogenation reactor 440 and the products sodium lactate and water subjected to vacuum distillation 455. Following vacuum distillation, the products may be subjected to electrodialysis 460. Ion exchange of the products obtained by electrodialysis leads to the formation of lactic acid which may be introduced to a hydrogenation reactor 475 or dehydration reactor 480 followed by vacuum distillation (490 or 495) to generate enriched n-propanol or enriched acrylic acid.

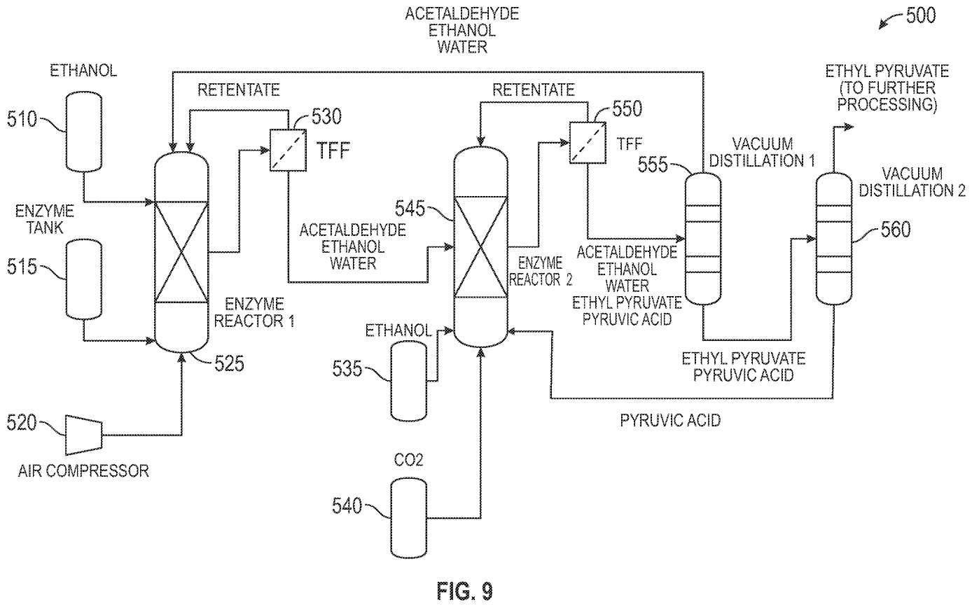

In yet other aspects of the present disclosure, ethanol is subjected to a plurality of enzyme reactors. For example, and with reference to FIG. 9, a process for HVC production 500 comprises introducing ethanol and an enzyme of the type disclosed herein are from storage tanks 510 and 515, respectively to enzyme reactor I 525. The product mixture exiting enzyme reactor I 525 comprises acetaldehyde, unreacted ethanol and water and can be conveyed through a TFF 530 to an enzyme reactor 2 545 where ethanol and carbon dioxide can be introduced from storage tanks 535 and 540, respectively. The product mixture from enzyme reactor 2 comprising acetaldehyde, unreacted ethanol, water, ethyl pyruvate and pyruvic may be passed through a TFF followed by vacuum distillation 555 to obtain final products or further processing.

### C4 Process Flow Diagram

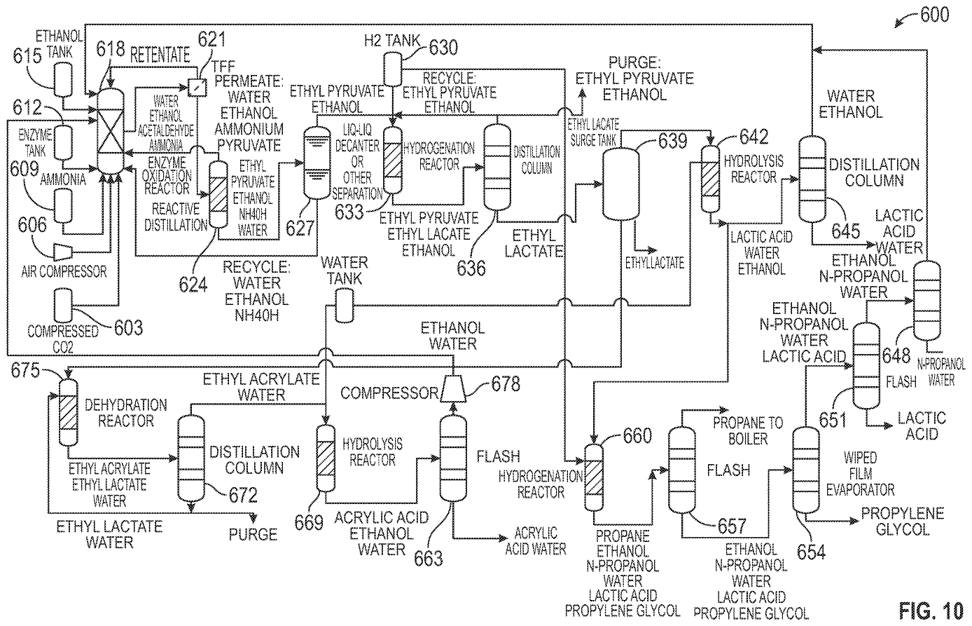

An exemplary process for the production of C4 compounds of the type disclosed herein is presented in FIG. 10, the enzyme oxidation reactor could be a sparged bubble column (840), an air lift column, a stirred sparged bioreactor, or a falling film high pressure oxidation vessel. Storage vessels 810, 820 and 830 may contain the reactants ethanol, enzyme and cofactor, respectively. The enzyme oxidation reactor may operate at temperatures ranging from about is 20° C. to 60° C. at pressures ranging from about 1 bar to about 15 bar. In the enzyme oxidation reactor 840, ethanol undergoes a multi-step enzymatic conversion first to acetaldehyde and then to acetoin, with cofactors present to aid the enzymatic conversion, and catalase present to degrade hydrogen peroxide for enzyme stability. The enzyme oxidation reactor 840 may be sparged with compressed air (for molecular oxygen). While not shown, pH can be controlled by the addition of strong acids, bases, or buffers. Enzyme reactor effluent may then be sent to a tangential flow filter (TFF) 845 to preserve enzymes in the enzyme reactor as recycled retentate, with permeate flowing further down the process.

Enzyme reactor effluent can then be distilled using a distillation column 850 to separate water, ethanol, and acetaldehyde from the acetoin product. Acetoin has by far the highest boiling point, at 148° C., and as such will be enriched in the bottoms. Distillate, containing water, ethanol, and acetaldehyde, may then be recycled to the enzyme oxidation reactor 840. This distillation will likely take place in at atmospheric pressure distillation column, though a single effect flash, an MVR, or a vacuum distillation may also be employed, depending on desired purity.

Purified acetoin can be withdrawn as a final product, or it can be hydrogenated over a metal catalyst to 2,3-butanediol. Hydrogenation of the purified acetoin may take place in a trickle bed reactor 860, with hydrogen introduced into the reactor feed line 855 as hydrogenation feedstock. Though not shown, temperature and pressure into the hydrogenation reactor will be controlled via feed pumps, compressors, and heat exchangers. The hydrogenation reactor can be operated in a downward gas-liquid trickle bed flow where the extrudates are on the order of 1 mm to 10 mm in primary length. The extrudates are fixed in place while the gas channels through the interparticle void space and the liquid flows as rivulets over the external support surface. With this catalyst, the hydrogenation reactor can be operated in slurry flow either in a plug flow regime where the liquid, gas bubbles, and catalyst are moving uniformly in a single direction. Alternatively, the reactor 860 can be operated in slurry flow in a well-mixed regiment via a mechanical agitator or using the gas to ebullate the slurry. In one or more aspects, the hydrogenation reactor 860 comprises a downward trickle bed. A mixture of 2-3 butanediol and unreacted acetoin are present in the reactor effluent.

This mixture can be separated via distillation. As acetoin (148° C.) and 2,3-butanediol (177° C.) are near-boiling, this will involve a fractional distillation column 865. Though the fractional distillation column 865 can operate at atmospheric pressure, purity requirements will likely necessitate the use of a vacuum fractional distillation to aid separation. Acetoin distillate is recycled back into the process, while 2,3-butanediol bottoms are recovered from the bottoms.

Production of 2,3-butanediol can be the end of this process, or 2,3-butanediol can flow further downstream to produce 1,3-butadiene. The 2,3-butanediol stream may have all trace water removed prior to dehydration. This may be achieved by vaporizing the 2,3-butanediol stream in a superheater and passing the stream through a molecular sieve column. Water is adsorbed onto the molecular sieve zeolites, and water-free 2,3-butanediol can continue to the dehydration reactor. Periodically, the molecular sieve will be regenerated by stripping the absorbed water from the zeolites. This is done by passing superheated steam through the bed, vaporizing the sequestered water. Alternatively, pervaporation may be used for water removal if the water content is high enough.

The 2,3-butanediol vapor can be heated to >300° C. prior to being conveyed to the dehydration reactor 875. In one or more aspects, disposed within the dehydration reactor 875 likely is a tantalum beta zeolite, hafnium beta zeolites, tin beta zeolites, a HfZn catalyst or combinations thereof. The dehydration reactor 875 may be operated at low conversions (10%-20%). In alternatively aspects, a hydroxyapatite-alumina catalyst may be disposed within the dehydration reactor 875.

Reactor effluent containing 2,3-butanediol and 1,3-butadiene may then fed to a distillation unit. Due to the wide boiling nature of the mixture (about 177° C. vs −4.4° C.), a single stage distillation, such as a flash, knockout drum, single effect evaporator, or mechanical vapor recompressor can be employed to separate the mixture. Heat integration, in the form of pre-heating the molecular sieve feed by condensing the reactor effluent, can be used to increase efficiency. Bottoms, containing 2,3-butanediol, can be recycled back into the process, while 1,3-butadiene distillate is recovered as product. While not shown, 1,3-butadiene can be compressed and or liquefied, as desired. Though not shown, 1,3-butadiene can undergo a further hydration reaction over a suitable hydration catalyst (e.g., ruthenium catalyst) to produce 2-butanone (methyl ethyl ketone or MEK).

Although the above drawing is at PFD level of detail, not all process interconnections are shown such as spillbacks, block and bleeds, recycle lines, control valves, cooling/heating elements, pumps, intermediate tankage, antifoam, etc.

In an aspect, the methodologies disclosed herein result in HVC compounds (e.g., C3 or C4 compounds) having a purity of greater than about 60%, alternatively greater than about 70%, alternatively greater than about 80%, alternatively greater than about 90% or alternatively from about 60% to about 95% using an in vitro production system. The compositions and methods of the present disclosure will generate C3 compounds at high purity using an in vitro system. Without wishing to be limited by theory, use of highly specific enzymes as catalysts eliminates or reduces the presence additional compounds and materials, facilitating separation and purification of the product and leading to lower production costs compared to traditional chemical synthesis. This benefit is particularly realized in the production of lactic acid as no fermentation steps are used which may introduce media and cellular components requiring extensive purification for removal. In another aspect, the methods and compositions disclosed herein result in production of C3 compound with reduced economic expenditures when compared to current methodologies for the production of HVC. For example, direct carboxylation of ethanol with carbon dioxide followed by selective partial hydrogenations at high space time yields represents the lowest feedstock cost basis pathway to highly pure C3 plastic monomers: lactic acid, acrylic acid, and propylene glycol.

In another aspect, the methods and compositions disclosed herein result in production of a HVC with improved safety when compared to current methodologies for the production of HVCs. For example, in the enzymatic steps, lower temperatures, and lower operating pressures (e.g., 100 psi in the enzymatic process) lead to an inherently safer process. The processes disclosed herein also use ethanol as a feedstock instead of propene and propanal feedstocks. Propene is not only flammable, but also can readily mix with other materials to produce explosives. Propanal is a respiratory irritant, air sensitive, and may form explosive peroxides upon prolonged storage. Propylene oxide is a probable human carcinogen. In another aspect, the methods and disclosed herein exhibit a reduced environmental impact when compared to current methodologies for the production of HVCs. For example, the processes disclosed herein use carbon dioxide as a substrate for pyruvate production. These factors result in the presently disclosed processes having a reduced output of carbon dioxide to combat anthropogenic climate change.

### Examples

The presently disclosed subject matter having been generally described, the following examples are given as particular aspects of the subject matter and to demonstrate the practice and advantages thereof. It is understood that the examples are given by way of illustration and are not intended to limit the specification or the claims in any manner.

### Mutant Enzyme

A mutated Fusarium graminearum GAO (FgGAO) and a native GAO homolog from Colletotrichum spinosum (CsAlcOX) has both been shown to be capable of oxidizing ethanol. The FgGAO sequence containing the M1 mutations and R330K, Q406T, and W290F, as well as the C383S mutation was able to oxidize ethanol with a specific activity of about 8 U mL⁻¹ E. coli lysate FIG. 11. Studies are underway to elucidate mutations in the FgGAO M-RQW-S scaffold to enhanced stability and activity.

### Example Protocol for Enzyme Testing

### Colorimetric Microtiter Plate Screening

Enzyme mutants will be screened using a microtiter plate-base colorimetric assay that monitors the production of hydrogen peroxide. For example, the reagents o-dianisidine and 2,2′-azino-bis(3-ethylbenzothiazoline-6-sulphonic acid) (ABTS) may be used in conjunction with horseradish peroxidase to elicit a color change in the presence of hydrogen peroxide. Enzymes or combination of enzymes are diluted to total stock concentration of 1 mg/mL and then diluted further into a roughly 200 μL volume containing glucose substrate, the reporter molecule, buffer, and horseradish peroxidase. The dilution factor is chosen such that the change in color falls within the range of 0.05-0.2 absorbance units per minute as measured with a plate-based spectrophotometer. This rate of change can be used along with the dilution factor and extinction coefficient of the reporter molecule to calculate the specific activity of the enzyme(s) or plotted to select mutants with high activity for further characterization.

### Parr Bomb Scale Testing

A pressurized Parr bomb system will be used to mimic the reactor conditions at scale in order to assess the efficacy of converting ethanol to acetaldehyde using the disclosed processes. In a final volume of 50 mL, enzymes at 1-0.001% w/v will be combined with 20% w/v ethanol and a buffer (typically phosphate buffer) at an initial pH of 4-8. Catalase may be added at a 1:1 to 1:20 EOX to catalase ratio to prevent accumulation of hydrogen peroxide. The mixture will be loaded into the Parr bomb containing a stir bar. To improve mass transfer of oxygen into the solution, the vessel will be sparged with oxygen two times, then pressurized to 100 atm. The reactor will be held at constant temperature, typically 20° C. but within the range of 10-80° C. and the mixture allowed to react until the reaction is complete. During the reaction, the vessel may be depressurized to adjust the pH and obtain samples to assess conversion and product profile. Testing methods may include HPLC for the detection of C3 compounds.

### Acetoin Hydrogenation to 2,3-Butanediol

To obtain 2,3-butanediol, acetoin generated from the enzymatic reactor will be hydrogenated over a ruthenium or other metal catalyst to generate the final product. Stereoselective catalysts may be used to generate the meso, (R,R), or (S,S) compounds.

### Dehydration of 2,3-Butanediol to 1,3-Butadiene Via Metal Catalysis

2,3-butanediol dehydration to form 1,3-butadiene may be performed using a metal oxide, zirconia doped with alkaline-earth elements, or rare earth orthophosphate catalyst. An element in ensuring high yield of butadiene is to limit pinacol rearrangement to yield butanone (methyl ethyl ketone, MEK) and isobutanol.

### Hydration of 1,3-Butadiene to 2-Butanone

The hydration of 1,3-butadiene to 2-butanone will be facilitated through use of a ruthenium catalyst. The catalyst may be synthesized from one equivalent of 1,10-phenathroline mixed with ruthenium trichloride.

### Prophetic Hydrogenation of Sodium Pyruvate to Sodium Lactate

As a prophetic example, hydrogenation is carried out in a stainless steel stirred autoclave. Reaction conditions: 1500 rpm stirring rate, ethanol (150 ml), Na-pyruvate (0.4 mol). 5% Pt/alumina (0.5 g) catalyst powder. Reaction temperature at room temperature at 70 bar. The catalyst is initially pretreated in hydrogen at 400° C. for 90 minutes. A conversion rate of 80% yields approximately 50% lactate formation.

### Prophetic Dehydration of Sodium Lactate to Acrylic Acid

As another prophetic example, a Y-type zeolite can be used as catalyst for alcohol dehydration. Lactic acid (LA. analytic grade) may be obtained from Sigma-Aldrich and deionized water is used for its dilution. The alkali phosphates (NaH₂PO₄, Na₂HPO₄, K₂HPO₄, and Na₃PO₄) and nitrates, together with hydroquinone are obtained from Sigma-Aldrich. NaY zeolite (SiO₂/Al₂O₃=2.5) is purchased from zeolith-bentonite purchase (www.zeolithe-bentonite.de). Hydroquinone (in appropriate amount) is used as polymerization inhibitor. To ensure complete Na change, the purchased NaY zeolite is treated in 1 mol/L NaNO₃ aqueous solution. The treated NaY zeolite is then impregnated with a selected phosphate. For example, 2.6 g of treated NaY is added into 25 mL aqueous solution of a desired phosphate of a concentration (corresponding to a desired loading). The mixture is stirred at 40° C. for 4.5 h, and the resulting slurry is subject to slow evaporation (in a rotary evaporator) at 62° C. until dryness. The obtained material is further dried at 125° C. overnight in an oven, and then pressed and crushed into particles of 40-60 mesh.

The evaluation of catalyst activity is carried out in an upright fixed-bed quartz reactor that is 8 mm in inner diameter and 500 mm in length. The reaction is operated at atmospheric pressure. A 1.5 g portion of catalyst is charged into the reactor, and the space above the catalyst bed is filled with quartz chips to ensure preheating of the in-coming LA-containing liquid. Before introduction of the feedstock, the sample is heated up in a flow of pure N2 (32 mL/min) to a desired temperature at a rate of 10° C./min and kept at this temperature for 3.5 h. Then a flow of LA liquid (3-10 mL/min) is introduced, and the products were collected at a cold trap. The analysis of the collected species is conducted using a gas chromatograph equipped with FID and HP-FFAP capillary. The performance test results in a conversion rate of 95% and a yield of acrylic acid of 25%.

### Prophetic Hydrogenation of Acetoin to 2,3 Butanediol

As another prophetic example, the catalyst (5% Pt on silica) is prepared in-house (Pt Aldrich, Silica Siralox 40, Sasol) by impregnation. Typically, the reactions are undertaken using the following procedure. The catalyst (0.2.5 g) and solvent (35 ml) are added to the HEL reactor. The reactor is then sealed and purged with hydrogen before heating to 62° C. while stirring the mixture at 800 rpm. Upon reaching 60° C. the reactor is held at this temperature and the mixture is stirred at 1000 rpm for 1.5 h to reduce the catalyst. Following the reduction of the catalyst, a solution of 4-phenyl-2-butanone (13.5 mmoles) in fresh solvent (20 ml) is added to the reactor containing the slurry of the pre-reduced catalyst. The HEL reactor is sealed and purged with hydrogen and the reactor is then heated to the reaction temperature, during which the mixture is stirred at 800 rpm. When the required temperature reaches typically 72° C., the stirring is stopped, and the reactor is pressurized. The reaction starts immediately upon reaching the required pressure (typically 5 bar) by starting the stirrer at 1400 rpm. The reactions are typically performed for 2.5 h, during which time regular reaction samples of about 2 ml are taken for analysis by GC. The rates are calculated by fitting a polynomial function to the concentration change with time and taking the differential of the curve at t=0. The performance test results in a conversion rate of 90% and a yield of 2,3 butanediol of 70%. See Na-pyruvate example exchange of Na to Ca (sodium to calcium)

### Prophetic Conversion of Ethyl Pyruvate to Ethyl-Lactate

Ir/SiO₂ is used as catalyst. The catalysts are prepared by wet impregnation of the support (SiO₂ BASF D-11-11, SBET=150 m²/g) with a toluene solution of iridium acetylacetonate, using the appropriate amount of iridium acetylacetonate to get 5 wt. % of Ir. The solids are calcined in air at 573 K and reduced in flowing H₂ at 500° C., prior the characterization or catalytic test.

The catalysts, reduced in situ prior each reaction, are dispersed in the reaction solvent to keep out of air. All the interacting components (catalyst, solvent, substrate and modifier) are fed into the stainless-steel batch reactor prior to the addition of hydrogen. In fact, this procedure is called premixing method. The reaction mixture is stabilized at the reaction temperature (25° C.) under a N₂ purge for few minutes and stirred at 900 rpm during all reaction-time. Then, H₂ is flashed to the reactor at atmospheric pressure to remove the inert gas and pressurized up to 42 bar. For standard experiments, the solvent used is cyclohexane (50 mL), and the weight of the catalyst is 100 mg. Liquid samples are taken periodically from the reactor and analyzed by a gas chromatograph-mass spectrometer (GCMS-QP5050 Shimadzu) provided with a chiral b-dex 225 column (30 m; Supelco). No side reactions, such as solvent dehydrogenation or hydrogenolysis reactions, are detected during the catalytic reaction. The performance test results in a conversion rate of 60% and a yield of ethyl-lactate of 50%.

In this prophetic example, 5 wt. % Ru/C is prepared through wet impregnation and sol-immobilization for hydrogenation of lactic acid to 1,2-propanediol. Reaction conditions of 120° C. 35 bar H₂. 2.5 h and 5% LA/H₂O with catalyst weight of 0.025 g are applied. Solutions of RuNO(NO₃)₃ for wet impregnation, and RuCl₃ for sol-immobilization are used as ruthenium precursors. The catalyst that is prepared through sol-immobilization technique showed higher conversion compared to the commercial Ru/C and wet impregnation catalyst. However, the sol-immobilization catalyst shows significant activation loss compared to the commercial catalyst. All the catalysts show approximately 100% 1,2-propanediol selectivity with conversions ranging from 10 to 25% (no recycle).

Ru coupled with MoOx on both carbon and silica are applied in hydrogenation of lactic acid to 1,2-propanediol. The Mo modified Ru on carbon catalyst shows four times higher TOF compared to Ru on carbon catalyst. The Ru-MoOx supported on silica shows lower activity compared to carbon supported catalyst. In both carbon and silica supported material Ru is in metal state and Mo valence was +4. Yield of 95% in 18 h at 393 K is achieved by Mo modified Ru on carbon catalyst Ruthenium on activated carbon is applied in hydrogenation of aqueous lactic acid to propylene glycol. Approximately full conversion is achieved at reaction temperatures of 100-170° C. and hydrogen pressures of 7-14 MPa over 8 hours.

While aspects of the presently disclosed subject matter have been shown and described, modifications thereof can be made by one skilled in the art without departing from the spirit and teachings of the subject matter. The aspects described herein are exemplary only and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the presently disclosed subject matter. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A chemoenzymatic manufacturing process for the preparation of high value chemicals, the process comprising:
enzymatic oxidation of ethanol using an oxidizing biocatalyst to form an acetaldehyde intermediate;
contacting of the acetaldehyde intermediate with a carboligating biocatalyst to form pyruvic acid;
reacting pyruvic acid with ethanol to form ethyl pyruvate;
contacting the ethyl pyruvate with a metal catalyst and hydrogen to form ethyl lactate;
hydrolyzing the ethyl lactate to produce lactic acid;
contacting at least a portion of the lactic acid with a dehydrating catalyst to form acrylic acid; and
contacting at least a portion of the lactic acid with a metal catalyst and hydrogen to form propylene glycol and n-propanol.

2. The process of claim 1, wherein the oxidizing biocatalyst comprises an alcohol oxidase, a copper-radical oxidase, variants thereof, fragments thereof, or combinations thereof.

3. The process of claim 1, wherein the oxidizing biocatalyst has any of SEQ ID NO: 1 through SEQ ID NO:67.

4. The process of claim 1, wherein the carboligating catalyst comprises a decarboxylase.

5. The process of claim 1, wherein the carboligating biocatalyst has any of SEQ ID NO:68 through SEQ ID NO:74.

6. The process of claim 1, wherein the hydrogenating metal catalyst comprises a transition-metal.

7. The process of claim 1, wherein the dehydrating metal catalyst comprises a transition-metal catalyst, a metal oxide, a cobalt and zinc catalyst, zirconia doped with alkaline-earth elements, a rare earth orthophosphate catalyst or combinations thereof.

8. A process of preparing a high value chemical, the process comprising:
introducing to a plurality of bubble reactors (i) an oxidizing biocatalyst (ii) a carboligating biocatalyst (iii) ethanol, and (iv) carbon dioxide under conditions suitable for the formation of pyruvate.

9. The process of claim 8, wherein the oxidizing biocatalyst has any of SEQ ID NO: 1 through SEQ ID NO:67.

10. The process of claim 8, wherein the carboligating biocatalyst has any of SEQ ID NO:68 through SEQ ID NO:74.

11. The process of claim 8, wherein the oxidizing biocatalyst comprises an alcohol oxidase, a copper-radical oxidase, variants thereof, fragments thereof, or combinations thereof.

12. The process of claim 8, wherein the oxidizing biocatalyst comprises a mutated galactose oxidase.

13. The process of claim 8, wherein the carboligating catalyst is a decarboxylase.

14. The process of claim 8, wherein the pyruvate has a purity of from about 60% to about 95%.

15. The process of claim 8, further comprising converting pyruvate to a compound selected from the group consisting of ethyl lactate; lactic acid, acrylic acid, propylene glycol, 1-propanol and 2-propanol.

16. A chemoenzymatic manufacturing process for the preparation of higher value chemicals from ethanol, the process comprising;
enzyme catalyzed oxidation and decarboxylation of ethanol to produce pyruvate;
subjecting the pyruvate to one or more processes selected from the group consisting of hydrolysis, dehydrogenation, oxidation, distillation, filtration and combinations thereof and recovering one or more products selected from the group consisting of lactic acid, acrylic acid, n-propanol, propanol, propylene glycol or combinations thereof.

17. The process of claim 16, wherein the enzyme-catalyzed oxidation is carried out in the presence of an alcohol oxidase, a copper-radical oxidase, variants thereof, fragments thereof, or combinations thereof.

18. The process of claim 16, wherein the enzyme-catalyzed oxidation is carried out in the presence of an enzyme having any of SEQ ID NO:1 through SEQ ID NO: 67.

19. The process of claim 16, wherein the enzyme-catalyzed oxidation and decarboxylation is carried in a multiphase enzyme reactor bioreactor comprising a gas phase, an aqueous liquid phase, an organic liquid phase, a hydrophilic solid phase with immobilized enzymes, a hydrophobic solid phase with immobilized enzymes or combinations thereof.

20. A chemoenzymatic manufacturing process for the preparation of C4 compounds, the process comprising:
enzymatic oxidation of ethanol using a oxidizing biocatalyst to form an acetaldehyde intermediate;
contacting of the acetaldehyde intermediate with a carboligating biocatalyst to form acetoin;
contacting acetoin with a metal catalyst and hydrogen to form 2,3-butanediol; contacting 2,3-butanediol with a metal catalyst to form 1,3-butadiene; and contacting 1,3-butadiene with a metal catalyst to form 2-butanone.

21. The process of claim 20, wherein the oxidizing biocatalyst comprises an alcohol oxidase, a galactose oxidase, variants thereof, fragments thereof, or combinations thereof.
